(19) United States
(12) Patent Application Publication
(10) Pub. No.: US 2005/0131739 A1
(43) Pub. Date: Jun. 16, 2005

(54) Methods for monitoring severity of panic attacks and other rapidly evolving medical events in real time

(75) Inventors: Joshua D. Rabinowitz, Mountain View, NJ (US); Roman Skowronski, Palo Alto, CA (US)

Correspondence Address: ALEXZA MOLECULAR DELIVERY CORPORATION, 1001 EAST MEADOW CIRCLE, PALO ALTO, CA, 94303, US

(73) Assignee: Alexza Molecular Delivery Corporation, Palo Alto, CA (US)

(21) Appl. No.: 10/996,114

(22) Filed: Nov. 23, 2004

Related U.S. Application Data
(60) Provisional application No. 60/530,059, filed on Dec. 15, 2003.

(51) Int. Cl. G06F 17/60; A61B 5/00

(52) U.S. Cl. 705/2

(57) ABSTRACT

The present invention provides novel methods of recording a medical event in real-time. One aspect of the invention comprises providing a subject an electronic diary for recording one or more aspects of a medical event, wherein the diary electronically records a time at which entries to the diary are made without requiring the subject to input the time of each entry and wherein the diary further prompts the subject to make an entry in the diary at various time intervals. Other aspects of the invention include methods of determining whether a medical treatment produces a therapeutic effect in a subject using real-time data; methods of measuring response of a subject experiencing panic attack symptoms to a treatment in real-time; and methods of assessing the relative effectiveness in real-time of a first medical treatment and a second medical treatment.

### REFERENCE TO RELATED APPLICATIONS

This application claims priority to U.S. provisional application Ser. No. 60/530,059 entitled “Methods for Monitoring Severity of Panic Attacks and Other Rapidly Evolving Medical Events in Real-Time,” filed Dec. 16, 2003, Rabinowitz and Skowronski the entire disclosure of which is hereby incorporated by reference.

### FIELD OF THE INVENTION

The present invention relates generally to the use of diaries in clinical trials. More specifically, the invention relates to the use of interactive electronic diaries for recording the severity of panic attacks and other rapidly evolving medical events in real-time.

### BACKGROUND

Diaries have provided valuable records of medical events for centuries. Although the applications for which diaries are used are extremely varied, ranging from recording highly personal thoughts to contributing to accurate inventory control, one important purpose of diaries is providing an accurate recounting of medical events, in particular medical events that evolve over days, weeks, or years. Diaries are considered uniquely valuable and accurate records of medical events spanning days, months, or years because entries are made in the diary many times during a medical event, generally daily, rather than post hoc after the medical event. In contrast to the unique presumption of the validity of diary entries made daily during medical events spanning many days, months, or years, diaries made post hoc after medical events are subject to the same biases as other post hoc accounts. This post hoc recounting bias is particularly important in considering diary records of rapidly evolving medical events occurring over minutes or hours instead of days or months, because it is difficult to ensure that diary entries are made during such brief medical events rather than after the fact. Thus, the presumption of validity that applies to daily diary entries of medical events spanning weeks or more does not necessarily apply to daily entries of medical events occurring in hours or less.

One specific use of diaries of substantial importance in the medical and pharmaceutical industries is recording of the effects of medical treatments, with diaries frequently used in studies that determine the effectiveness of drugs. These self-reported diaries have been used to collect information on many subjective and behavioral variables in clinical studies, including chronic pain, headache, urinary incontinence, etc. The results of such studies not only inform medical practice, they also may determine whether drugs gain approval from regulatory bodies such as the U.S. Food and Drug Administration. Thus, such diary records have profound economic consequences. A problem with such records, however, is the tendency for patients to record diary entries post doc, sometimes just before a patient returns a diary to a physician or other medical personnel, rather than during the medical event of interest. This problem is particularly severe for acute and episodic medical events. Because of this problem, adequate studies of potential treatments for certain short-lived conditions such as acute panic attacks have not been conducted, and adequate treatments have not been identified.

One effort to remedy the problem of diary records of medical events being reconstructed post hoc is the use of electronic patient diaries in recording medical events. The recent advent of the electronic hand-held diary, in which the user makes a record not on paper but in an electronic medium such as a computer, has greatly simplified and facilitated the acquisition of data in a diary. Typically, the electronic diary includes a display device to display textual information and a user interface which allows a user to navigate through the textual information and access various features of the device. Generally, the display device and user interface are incorporated in a hand-held housing to facilitate portability of the device. Additionally, the electronic diary has the ability to accurately record the time of entry, thus facilitating the assessment of the reliability and applicability of the data entered. Such diaries can provide a time stamp for entries, ensuring that the entry and the medical event occur in temporal proximity, and avoiding back-dating of entries as frequently occurs in traditional paper diaries.

Focusing specifically on the recording of panic attacks, an electronic diary has been used to record the time at which patients experience acute panic, and the intensity of the attack overall. The diary has also been used to check on an hourly basis for the presence of panic in subjects known to have panic disorder (Taylor, C. B., Fried, L., and Kenardy, J. Behav. Res. Ther. 28: 93-97). Such diaries, however, have not been used to monitor the progression of a particular panic attack. Indeed, because of the lack of adequate means to measure the course of particular panic attacks in the field, the natural history of panic attacks remains poorly defined.

The present invention provides the methods for measuring not only the course, but also various aspects of rapidly evolving medical events in the field by using a new data collection method and demonstrates for the first time the use of such methods in tracking in real-time, i.e., during the event, the severity and duration of an acute medical event such as panic attack in several subjects. The methods of the invention can also be used to determine whether an on-demand treatment would be effective for treating panic attacks, as opposed to relying solely on chronic treatment methodologies for controlling panic disorders.

### SUMMARY

The present invention provides methods for recording, during the course of a medical event, aspects of the medical event in a diary in real-time. In certain embodiments of the invention, a method of recording a medical event in real-time is provided that comprises providing a subject an electronic diary for recording one or more aspects of the medical event, wherein the diary electronically records a time at which entries to the diary are made without requiring the subject to input the time of each entry and wherein the diary further prompts the subject to make an entry in the diary at least twice within every 54 minute time period, following activation of the diary by the subject upon the initiation of the medical event, and continuing until deactivation of prompting from the diary. In other embodiments, the diary prompts the subject with yet greater frequency, encouraging recording of multiple entries during a medical event of even brief duration. In some embodiments, at least one aspect of the medical event is recorded on a scale, sometimes a visual analogue scale.

Certain other embodiments of the present invention focus on the recording of a medical event such as a panic attack in real-time. One such embodiment involves a method comprising providing a subject an electronic diary for recording one or more aspect of the panic attack, wherein the subject makes an entry in the diary frequently, such as at least three times within a 36 minute time period, or yet more frequently. In some embodiments, the diary prompts the subject to make entries. In certain methods, the entry in the diary comprises a score of a scale, wherein the score indicates the severity of the subject's panic symptoms.

In yet other embodiments, the present invention applies the above methods for recording, during the course of a medical event, aspects of a medical event in a diary by entering at least one measure of symptoms being experienced during the event, to provide methods for determining whether a medical treatment produces a therapeutic effect in a subject. In certain embodiments, the medical treatment is a drug, and the subject self-administers the drug, sometimes by a route of administration resulting in rapid achievement of maximal plasma drug concentrations such as by inhalation.

In a related aspect, the present invention applies the above methods for recording of a medical event such as a panic attack in real-time to provide methods of measuring the response of a subject experiencing panic attack symptoms to a treatment. In certain embodiments, the treatment is a drug in the form of a small particle size aerosol administered by inhalation. In certain embodiments, the drug has sedative properties related to enhancing of GABA-type neurotransmission. Benzodiazepines, such as alprazolam and clonazepam, are such drugs.

In yet another related aspect of the present invention, the invention provides methods for using the above and related methods for recording of a medical event in real-time to provide methods of assessing the relative effectiveness of a first medical treatment and a second medical treatment. These methods comprise measuring the response to the first and second treatments using the above or related methods, and then comparing diary entries made after administration of the first medical treatment with entries made after administration of the second medical treatment. In certain embodiments, the first medical treatment comprises a placebo and the second medical treatment comprises a drug, and subjects are randomly assigned to receive drug versus placebo. In certain embodiments, statistical tests are used to compare the responses to the treatments.

### BRIEF DESCRIPTION OF THE DRAWINGS

FIG. 1 is a photograph of one commercially available electronic patient diary with a visual-analogue scale shown on the screen of the electronic diary.

FIG. 2 is a photograph of the electronic patient diary described in Example 1 at the stage where the diary is prompting the patient to enter a patient password.

FIG. 3A is a photograph of the electronic patient diary described in Example 1 at the stage where the diary is prompting the patient to enter the severity of the patient's panic symptoms on a visual-analogue scale.

FIG. 3B is a photograph of the electronic patient diary described in Example 1 at the stage where the patient has just entered the severity of the patient's panic symptoms as “10” on the visual-analogue scale.

FIG. 4 is a photograph of the electronic patient diary described in Example 1 at the stage where the patient has just entered the time since taking the patient's medication.

FIG. 5 is a photograph of the electronic patient diary described in Example 1 at the stage where the diary is prompting the patient to turn the diary on so that the patient can make an entry.

FIG. 6 is a photograph of the electronic patient diary described in Example 1, at the stage post hoc of the event, where the diary is prompting the patient to enter the greatest level of severity of heart palpitations, pounding heart, or accelerated heart rate experienced by the patient during the panic attack.

FIG. 7A shows the time course of panic attacks experienced by Subject 1 of Example 2 with diary entries made every 5 minutes.

FIG. 7B shows the time course of panic attacks experienced by Subject 1 of Example 2 with diary entries made every 2 minutes.

FIG. 8A shows the time course of panic attacks experienced by Subject 2 of Example 2 with diary entries made every 5 minutes.

FIG. 8B shows the time course of panic attacks experienced by Subject 2 of Example 2 with diary entries made every 2 minutes.

FIG. 9A shows the time course of panic attacks experienced by Subject 3 of Example 2 with diary entries made every 5 minutes.

FIG. 9B shows the time course of panic attacks experienced by Subject 3 of Example 2 with diary entries made every 2 minutes.

FIG. 10 shows the time course of panic attacks experienced by Subject 4 of Example 2 with diary entries made every 2 minutes.

FIG. 11 shows the average ±SE of the time course of panic attacks experienced by Subject 1 of Example 2 with diary entries made either every 2 minutes or every 5 minutes

FIG. 12 shows the average ±SE of the time course of panic attacks experienced by Subject 3 of Example 2 with diary entries made either every 2 minutes or every 5 minutes

### DETAILED DESCRIPTION

Definitions

The term “alprazolam” as used herein refers to 8-chloro-1-methyl-6-phenyl-4H-[1,2,4]triazolo[4,3-α] [1,4]benzodiazepine, which has the empirical formula of C₁₇H₁₃ClN₄, and includes any of its pharmaceutically suitable salts and any formulation or composition comprising said compound and/or its salts.

The term “aspect” as used herein with respect to a medical event refers to a characteristic or distinct feature of the medical event. For example, in the case where the medical event is a panic attack, an aspect of the medical event could include the severity of the panic attack or the presence, absence, or severity of a particular panic attack symptom.

The term “clonazepam” as used herein refers to 5-(2-chlorophenyl)-1,-3-dihydro-7-nitro-2H-1,4-benzodiazepin-2-one, which has the empirical formula of C₁₅H₁₀Cl₃N₃O₃, and includes any of its pharmaceutically suitable salts and any formulation or composition comprising said compound and/or its salts.

The term “diary” as used herein refers to a record, especially a personal record of events, experiences, and observations. Preferably, the diary involves entries made in a written form by the person experiencing the medical event first hand, although in certain instances it is acceptable for the entries themselves to be made by a scribe acting in response to instructions from the person experiencing the medical event first hand, for example a nurse recording the responses of a patient. Preferred forms of diary entries include not only hand writing but also typing, writing into a computer or other electronic instrument, and the like, and handwritten, typed, or electronic responses on scales.

The term “drug” as used herein refers to any substance that is used in the prevention, diagnosis, alleviation, treatment or cure of a condition. The terms “drug” and “medication” are herein used interchangeably.

The term “event” as used herein refers to something that happens at a given time.

The term “medical treatment” or “treatment” as used herein refers to an approach for obtaining or attempting to obtain a therapeutic effect and can include a placebo treatment. Although placebo alone is not a preferred medical treatment in the context of the present invention, when a placebo is used in a clinical trial as a comparator for an active substance or preparation, the placebo constitutes a “medical treatment” of the present invention, because the placebo is used in the trial to attempt to obtain a therapeutic effect, for the purposes of comparing the therapeutic effect produced by the placebo to that produced to the active treatment. Thus, in the context of methods of the present invention that compare two or more medical treatments, one or more of the medical treatments may be a placebo treatment.

The term “measure” refers to an amount or quantity or relative amount or quantity on a scale or a degree of, and/or presence or lack of whatever is being measured.

The term “placebo” as used herein refers to an inactive substance or preparation used as a control in an experiment to determine the effectiveness of a medicinal drug.

The term “scale” as used herein refers to a standard of measurement, preferably comprising one or more of the following: a range from a lower number to a higher number (e.g., severity of symptom(s) from 1 to 10), a range between two extremes (e.g. from no pain to the worst pain of one's life), graduations, a continuum on a line between two endpoints, lines at regular intervals, or a series of questions and/or categories with a score associated with certain responses and/or categorizations, or a combination of two or more of the above.

The term “score” as used herein refers to a value, typically a numerical value typically on a scale.

The term “sedative” as used herein refers to an agent or a drug having a soothing, calming, or tranquilizing effect.

The term “severity” as used herein refers to the extent or power of a distressing condition, symptom, or group of symptoms.

The term “symptom” as used herein refers to any sensation or change in bodily function experienced by a subject, generally such sensations or changes in bodily function that are associated with a particular disease or disease-related medical event

The term “therapeutic effect” as used herein refers to beneficial or desired clinical results including, but are not limited to, one or more of the following: alleviation of symptoms (whether partial or complete), diminishment of extent of a disease, stabilization (i.e., not worsening) of a state of disease or symptoms of disease, preventing spread (i.e., metastasis) of disease, preventing occurrence or recurrence of disease, delay or slowing of disease progression, amelioration of the disease state, and remission (whether partial or total).

The term “visual-analogue scale” as used herein refers to a scale comprising a positional component and a numerical component. The positional component generally comprises an axis with lower responses on one end of the axis and higher responses on the other end of the axis. The higher response end of the axis may be in any orientation with respect to the lower response end, but most commonly would be placed directly above or directly to the right of the lower response end of the axis. The numerical component generally comprises a series of consecutive integers, most commonly ranging from a minimum of 0 or 1 to a maximum of 3, 4, 5, 6, 7, 8, 9, 10, 99, or 100. Although, the aforementioned integers will commonly be used, it is readily apparent to one of skill in the art that any numerical value, whether whole or partial integers, fractions, percents, etc., and any range of numerical values can be used without diverging from the scope of the invention described herein. In some embodiments, the numerical component is not limited to integers and may be calculated based on a solely positional response. For example, a user might mark a point on an axis consisting of a line segment with severe symptoms at one end of the line segment and no symptoms at the other end of the line segment, with the location of the marked point indicative of the severity of the user's symptoms, and the numerical component of the visual-analogue scale could be calculated (sometimes without explicit knowledge of the user) by directly measuring the distance (for example in millimeters) between the marked point and one end of the line or the software can calculate the distance automatically and assign a relative numerical value to it.

Unless otherwise indicated, all numbers expressing quantities of ingredients, reaction conditions, and so forth used in the specification and claims are to be understood as being modified in all instances by the term “about.”

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting.

The section headings used herein are for organizational purposes only, and are not to be construed as limiting the subject matter described. All documents cited in this application, including, but not limited to patents, patent applications, articles, books, and treatises, are expressly incorporated by reference in their entirety for any purpose.

Diaries for the Frequent Recording of Aspects of Medical Events

The present invention provides methods of using diaries of particular utility in frequent recording of aspects of medical events. These methods of using diaries have features that render them particularly reliable for recording medical events, especially medical events that occur over one to several hundred minutes. While any form of diary can be used in the methods of the present invention, typically the diaries used are electronic. Numerous electronic devices capable of serving as diaries for the purposes of the present invention are commercially available. These include virtually any form of personal computer on the market. For example, using a Dell Dimension 2400 with 2.4 GHz Intel Celeron Processor (www.dell.com) running Microsoft Windows software including Microsoft Word it is possible to use the computer as an electronic diary by recording entries by typing into a Microsoft Word document. The entries can then be saved in a directory on the computer, with a timestamp automatically attached to each entry. Time on the computer can be set, for example, by entering the time into the computer manually or by connecting the computer to a server whereby the computer then learns the time from the server. Once the time on the computer is set, then the timestamp generated by Microsoft Windows and/or Microsoft Word automatically provides the correct time of each entry without the need for the diary user to input the time. While the above approach provides an exceptionally straightforward means of making electronic entries that serve as an electronic diary, it is less than optimal because the computer is bulky, the security of the diary entries and their associated time stamps may not be optimal, the Microsoft program may not provide adequate prompts to the user to make entries in the diary, and more directed entries than free form writing into a Microsoft Word document may be preferable.

Currently, any concerns about the bulkiness of computers as electronic diaries are adequately addressed by numerous electronic devices that are commercially available. One practical and convenient type of electronic device for use in the present invention is a personal digital assistant. For example, the Palm i705 (www.palmone.com) provides 33 MHz of computing power and 8 MB of RAM in a less than 0.25 kg device. Preferred electronic diaries weigh at least 0.01 kg and less than 2 kg, 1 kg, 0.5 kg, or 0.25 kg. Personal digital assistants can readily be programmed to record virtually any form of diary entry, including entries comprising virtually any type of scale (see below).

Whether using a personal computer, personal digital assistant, or any other form of device, electronic or not, it is advantageous to have a secure means of recording entries in the diary. For electronic means of making entries, such security is generally provided by selection or writing of appropriate software using approaches known in the art. For use of data from the diary to gain approval to market drugs, appropriate diary entry security may be a requirement of the regulatory agency reviewing the data. Numerous forms of software known in the art provide appropriate entry security. For example, PHT Corporation of Charlestown, Mass. (www.phtcorp.com) provides equipment that allows secure recording (with timestamps) in a manner that the U.S. Food and Drug Administration has found acceptable and that complies with the Unites States Code of Federal Regulations 21 CFR Part 11. See U.S. Pat. Nos. 5,778,882 and 6,095,985 to Raymond et al.

Beyond providing secure recording of entries, in certain methods of the present invention it is important in some aspects of the invention for the diary to prompt the user to make an entry. Such prompting, in particular frequent prompting, is key in some embodiments of the methods of the present invention to enabling highly reliable recording of medical events occurring over relatively brief periods of time. The prompt provided by the diary to the user (or subject) can take many forms; for example, the prompt may be visual, auditory, tactile, gustatory, or olfactory, generated by methods known in the art. Most computers and personal digital assistants, when appropriately programmed using standard programming approaches, can produce visual and auditory prompts. Most telephones provide auditory prompts. Simple technologies also commonly used in commercial products, sometimes personal digital assistants but more commonly pagers or cell phones can provide tactile prompts. A cell phone or pager in vibration mode is an example of one such tactile prompt, although the tactile prompt may consist of any of numerous types of tactile sensation. For example, the tactile prompt could consist of a temperature prompt. Technologies for converting electric energy into heating or cooling are well known. Gustatory and olfactory prompts are less well known, but also feasible using technologies such as those described in U.S. Pat. No. 6,602,475 to Chiao or by placing a small flavor reservoir in the mouth wherein the reservoir ejects (for example, using a pump) drops of flavor as the prompt.

In certain methods of the present invention, another aspect of the diaries that enables highly accurate records to be made is appropriate programming of the recording device to guide the subject making the entries. That is, the subject does not make free form entries but entries that conform to a particular format. These formats may consist of making entries on scales. One simple method to make such entries is for the subject to use a keypad or keyboard to type in the entry of the scale, for example to type the number “4” to indicate a particular response on a scale from 0 to 10. Another simple method is to use a stylus to touch on a screen a particular response, for example to select the number “4” off of a screen showing, for example, numbers from 0 to 10 on a visual analogue scale. In other embodiments of the invention, the subject may input responses to the diary via any means known in the art, including but not limited to use of a telephone to enter responses by dialing or speaking, or use of voice recognition software to handwriting software to record entries in a computer or personal digital assistant.

In certain embodiments of the present invention, the diary may be on continuously. In some such embodiments, the diary may provide prompts continuously. In other embodiments, however, for the purposes of saving diary power or avoiding providing unnecessary prompts when medical events of interest are not occurring, or for other reasons, the diary may shut off or not provide prompts until triggered to do so by a stimulus. The diary may be programmed to become activated in response to any of a variety of stimuli. Exemplary stimuli are as follows:
(a) a particular time of day or of the week;
(b) conscious input from the user that a medical event of interest is occurring or likely to occur in the near future (e.g., the user pushing a start button or flipping an “on” switch, or the user by responding to one or more specific questions in the diary, which are programmed such that when responded to, or responded to in a specific manner, activate the prompt);
(c) unconscious input from the user indicative of a medical event of interest (e.g., detection using any detector of physiology or pathophysiology of the user of a change in the physiology or the user; for example, the detector may continuously montitor breathing pattern or cardiac function and signal the diary to begin recording in response to an increase in heart rate or depth of breathing; appropriate tools for such detection are described in U.S. Pat. Nos. 6,413,224 and 6,551,252 to Sackner et al.
(d) an environmental medical event, such as a vibration, a flash of light, a sound, or the like.

Likewise, deactivation of a prompt from the diary or shutting off of the diary can be triggered using the stimuli mentioned above, with the exception that unconscious input or the conscious input from the user is indicative of completion or termination of the medical event of interest (e.g., a decrease in heart rate ,or the user pushing an off or shutdown button or flipping an “off” switch, or the user responding to one or more questions that indicate that the event as been terminated, as for example only, the prompt can be automatically deactivated once the subject has entered a 0 score three times consecutively regarding the severity of the medical event). Alternatively, deactivation may occur when the device runs out of power, the user stops responding, or at some predetermined time.

Frequency of Diary Entries

A key distinguishing feature of the methods of the present invention is the frequency with which prompts are provided to the diary user and/or diary entries are made by the diary user. The motivation for such frequent recording of entries was the inventors recognition that such recording, previously considered impractical or not useful, could be of great value in understanding certain medical events, in particular medical events of brief duration, especially when these medical events could be better characterized by diary records of the subjective experience of the subject experiencing the medical event (perhaps combined with physiological records) than by physiological records alone. A particularly important example of such a medical event is a panic attack, as described in greater detail below.

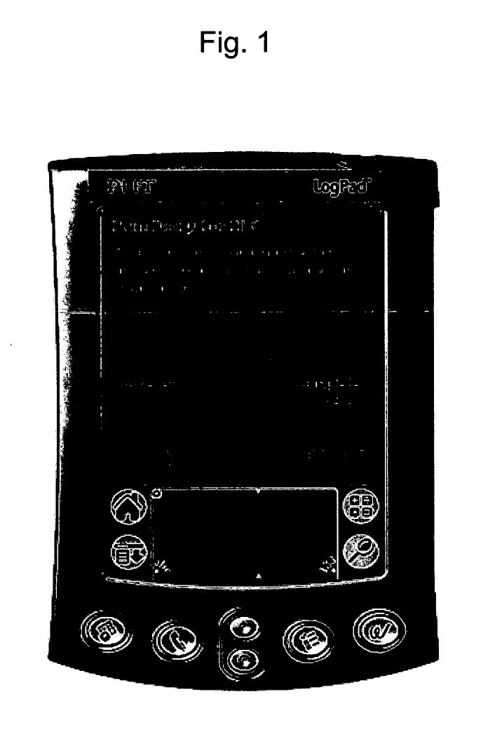

Prior to design of practical user interfaces for making frequent diary entries (for a particular example see FIG. 1) and prior to conducting experiments on subjects of their ability to make such frequently entries, it was not known whether such frequent measurement would be practical, i.e., would a subject be able to comply with such frequent recording of diary entries during a medical event. Further it was not known whether prompts from the diary would be useful in producing such recording of entries. Further it was not known whether such frequent recording of the severity of symptoms of a medical event would, through the very process of recording the medical event, so perturb the natural history of the medical event as to render the recording of no or minimal practical value. Finally, with particular respect to panic attacks, it was not known whether patients experiencing panic attacks or symptoms thereof would be capable of making diary entries during the attack.

It is the finding of the inventors that subjects indeed can comply with frequent diary prompts to make useful diary entries. In certain methods of the present invention, these prompts occur at least in a certain number per certain time interval, as follows:
(a) at least 2, 3, 4, 5, 6, 9, 10, 12, 15, 18, or 20 times per 54 minute time period;
(b) at least 2, 3, 4, 5, 6, 9, 10, 12, 15, 18, or 20 times per 36 minute time period;
(c) at least 2, 3, 4, 5, 6, 9, 10, 12, 15, 18, or 20 times per 24 minute time period;
(d) at least 2, 3, 4, 5, 6, 9, 10 ,12, 15, 18, or 20 times per 18 minute time period;
(e) at least 2, 3, 4, 5, 6, 9, 10, 12, 15, 18 or 20 times per 12 minute time period;
(f) at least 1, 2, 3, 4, 5, 6, 9, 10, or 12 times per 6 minute time period.

In certain methods of the present invention the number of prompts in a certain time interval falls in the following ranges:
(a) at least 2, 3, 4, 5, 6, 9, 10, 12, 15, 18 or 20 times and not more than 27, 54, or 108 times per 54 minute time period;
(b) at least 2, 3, 4, 5, 6, 9, 10, 12, or 15 times and not more than 18, 36, or 72 times per 36 minute time period;
(c) at least 2, 3, 4, 5, 6, 9, 10 times and not more than 12, 24, or 48 times per 24 minute time period;
(d) at least 2, 3, 4, 5, or 6 times and not more than 9, 18 or 36 times per 18 minute time period;
(e) at least 2, 3, 4, or 5 times and not more than 6, 12, or 24 times per 12 minute time period
(f) at least 1 or 2 and not more 3, 6, or 12 times per 6 minute time period.

In certain methods of the present invention, the diary prompts the subject to make entries at a pre-determined time interval, wherein the time interval is sufficiently frequent so as to allow accurate recording of even relatively brief medical events, such as panic attacks, without relying on post hoc recounting of the course of the medical event, and thus avoiding the inaccuracies associated with such post hoc recording. In certain such methods, the diary prompts the subject to make entries at a pre-determined time interval of less than or equal to every 18, 15, 12, 10, 6, 5, 4.5, 3, 2, 1.5, or 1 minutes. In certain embodiments, the diary prompts the subject to make entries no more frequently than every, 0.25, 0.5, 0.75, 1, 1.5, or 2 minutes. In some embodiments of the methods of the invention, the prompting begins only after the diary receives a signal from the user that a medical event is occurring is about to occur. In certain such methods, the signal involves a conscious action of the user.

It is a further finding of the inventors that subjects can make quite frequent diary entries, even during distressing medical events such as a panic attack, and that these entries can have value in describing the medical event. In certain methods of the present invention, the subject makes an entry in the diary at least a certain number of times in a certain time interval, for example:
(a) at least 2, 3, 4, 5, 6, 9, 10, 12, 15, 18, or 20 times per 54 minute time period;
(b) at least 2, 3, 4, 5, 6, 9, 10, 12, 15, 18, or 20 times per 36 minute time period;
(c) at least 2, 3, 4, 5, 6, 9, 10, 12, 15, 18, or 20 times per 24 minute time period;
(d) at least 2, 3, 4, 5, 6, 9, 10, 12, 15, 18, or 20 times per 18 minute time period;
(e) at least 2, 3, 4, 5, 6, 9, 10, 12, 15, 18 or 20 times per 12 minute time period;
(f) at least 1, 2, 3, 4, 5, 6, 9, 10, or 12 times per 6 minute time period.

In certain methods of the present invention, the number of entries that the subject makes in the diary in a certain time interval falls in the following ranges:
(a) at least 2, 3, 4, 5, 6, 9, 10, 12, 15, 18 or 20 times and not more than 27, 54, or 108 times per 54 minute time period;
(b) at least 2, 3, 4, 5, 6, 9, 10, 12, or 15 times and not more than 18, 36, or 72 times per 36 minute time period;
(c) at least 2, 3, 4, 5, 6, 9, 10 times and not more than 12, 24, or 48 times per 24 minute time period;
(d) at least 2, 3, 4, 5, or 6 times and not more than 9, 18 or 36 times per 18 minute time period;
(e) at least 2, 3, 4, or 5 times and not more than 6, 12, or 24 times per 12 minute time period
(f) at least 1 or 2 and not more 3, 6, or 12 times per 6 minute time period.

In certain methods of the present invention, the subject records an entry in the diary at least once every pre-determined time interval. In certain such methods, the subject records an entry at least once every 18, 15, 12, 10, 6, 5, 4.5, 3, 2, 1.5, or 1 minutes.

While it is possible in the above methods that the diary continues prompting the subject indefinitely, e.g., until the diary no longer functions, or that the subject continues making entries in the diary indefinitely, e.g., for the lifetime of the subject, in preferred methods the diary terminates prompting or the subject stops making entries within some reasonable length of time after initiating such an activity, as the advantages of the methods of the present invention are most profound for recording of relatively brief medical events. Thus, in certain embodiments methods of the present invention the subject may make entries or the diary may generate prompts for every minute or multiples or fractions thereof for at least 3, 6, 9, 12, 24, 36, or 54 minutes and not more than 1, 2, 4, 8, 12, 16, or 24 hours. In more preferred methods, the duration of making entries or generating prompts is tailored to the duration of the medical event being recorded. For example, in the recording of a panic attack the diary may provide prompts or the subject may record entries for a duration consisting of the duration of the panic attack. In certain embodiments, if the panic attack persists for greater than some period of time, for example greater than 30, 60, 90, or 120 minutes the subject may stop recording or the diary may stop generating prompts at the end of that time period even though panic is ongoing. In measuring panic attacks or symptoms thereof, one useful signal that the attack has terminated is the patient indicating to the diary that they are no longer experiencing any or significant panic symptoms. In certain methods of the present invention, the diary must receive such input from the subject several times, e.g. 2, 3, 4, or 5 times before the diary stops prompting the subject to make entries in the diary. In other methods, the subject makes at least 1, 2, 3, 4, of 5 entries of no or minimal panic, or a zero score on severity of panic attack symptoms, prior to terminating making entries of a particular panic attack.

The above description of termination of recording of a medical event in no way is meant to describe termination of utility of the diary. That is, one diary may be used to record one or many medical events, thus the diary retains utility after completion of recording of one particular medical event. In addition, in certain methods the diary may be used to record overlapping medical events.

Prompts

The technical features of a diary required to enable generation of prompts are described above, as are appropriate frequencies of prompting for use in methods of the present invention. As described above, prompts may be auditory, visual, tactile, gustatory, or olfactory. One preferred prompt is a brief noise, for example, a simple beep lasting less than 5 seconds. Preferably, the noise is at a comfortable but clearly audible volume, and effective to alert the subject making records in the diary of the need to make a new record without scaring or jarring the subject. In certain methods of the present invention, the prompt may repeat itself until the subject acknowledges the prompt, for example, beeping every 10 seconds or continuously making a noise until the prompt is acknowledged by the subject. In certain methods, the prompt may become louder over time until it is acknowledged. Beyond noises, a variety of other prompts are appropriate for use in the methods of the present invention, such as a mechanical vibration or other mechanical prompt, or a visual prompt such as a light flashing or turning on. Combinations of prompts may also be used.

When focusing on prompts used in the measurement of medical events, it is important that the prompts are sufficient to gain the attention of the patient even as the medical event is occurring, yet also not so alarming as to alter the natural history of the medical event substantially, nor so alarming as to render the diary overly embarrassing for the patient to use. The latter concern is especially important for diaries that may be used in public.

Scales

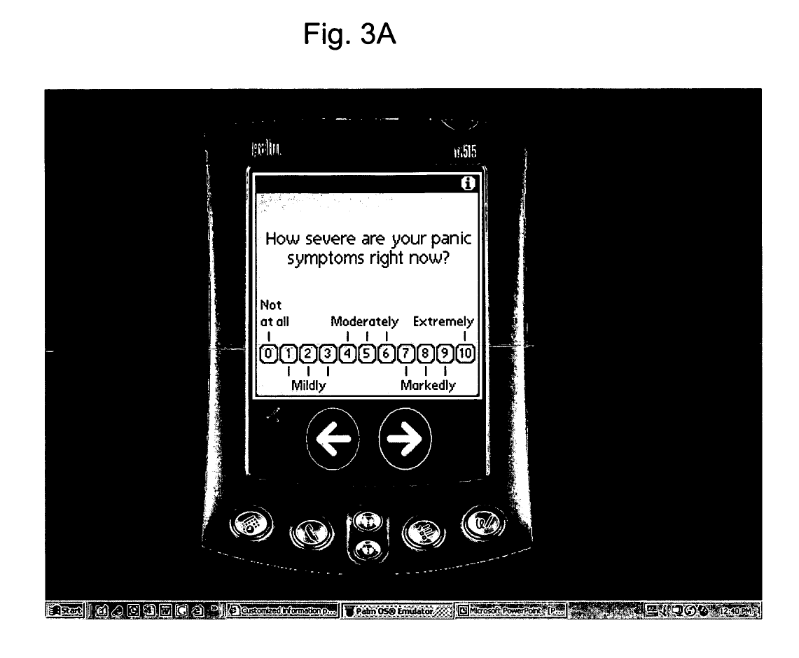

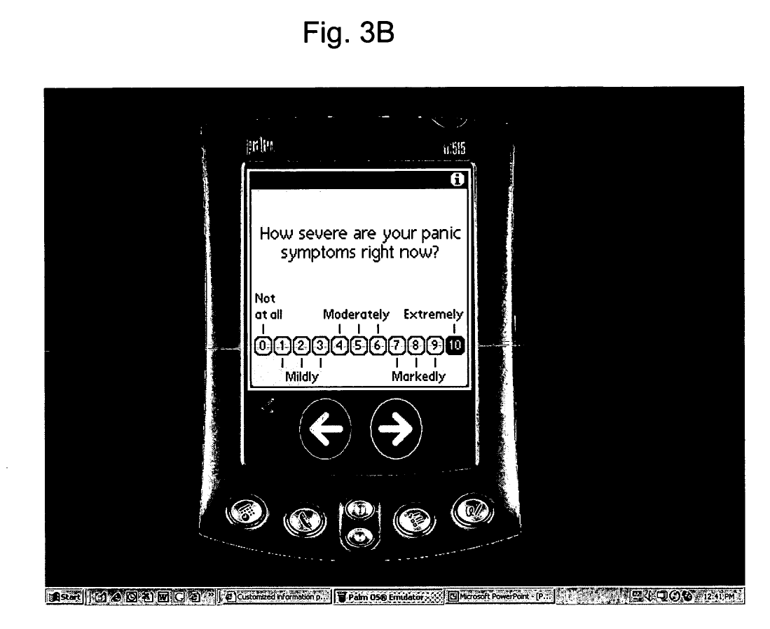

In certain embodiments of the present invention entries made in the diary comprise scales. Particularly useful are visual-analogue scales. Examples of visual-analogue scales appropriate for use in the methods of the present invention is shown in FIGS. 1 and 3. The scale shown in FIG. 1 shows a line segment defining an axis, with no relief at one end of the line segment and complete relief at the other end of the line segment. The patient uses a stylus or other marking tool to indicate a position (point) on the line segment which best describes the extent to which the study drug has provided relief. A numerical value can then be assigned by the electronic diary to the position of the mark, with the numerical value directly proportional to the distance between the mark and a reference end of the line segment.

The scale shown in FIG. 3 shows numerical values arranged on an axis, with verbal descriptors at both ends of the scale and at middle points in the scale. All of the above clues facilitate the subjects use of the scale, and accuracy of such use even when distracted by medical events such as panic attacks or symptoms thereof. Certain visual-analogue scales may have specific numbers of discrete integer responses, herein referred to as points, for example a scale having possible responses of 0, 1, 2, 3, 4, 5, 6, 7, 8, 9, and 10 would have 11 points. Preferred visual analogue scales have been 4 and 101 points, more preferably between 6 and 12 points, and most preferably 11 points.

When using scales with a numerical component, choice of the numbers to place on the scale or numbers within which answers on the scale may range is of importance in facilitating accurate recording of medical events. Generally, responses limited to integers are preferred, although real numbers are also possible. Preferred integers for responses fall in the range from −100 to 100, more preferably from 0 to 100, and yet more preferably from 0 to 10. Scales can also involve small numbers of integers, for example, 0 and 1 or 1 and 2 or 0, 1,2, and 3, or 1, 2, 3, and 4.

In addition to simple visual-analogue scales, numerous other scales for the measurement of different medical events are known in the art. Of particular utility for methods of the present invention are scales, for the measurement of panic attacks, symptoms thereof, or other medical conditions having a psychiatric, neurological, or other central nervous system component. Such scales are known in the art, with a variety of useful scales described by J. D. Guelfi in Clinical Neuropharmacology volume 11, supplement 2, pages S59-S68 (1988). Examples of use of various scales to evaluate the responses of patients with panic to drugs are described by G. M. Asnis et al. in Psychiatry Research volume 103, number 1, pages 1-14 (2001). All scales or batteries described in Guelfi above or Asnis et al. above are hereby incorporated in their entireties by reference. Further incorporated by reference are the Panic and Anticipatory Anxiety Scale of Sheehan, the Clinician Rated Global Improvement Scale of Sheehan, the Global Severity of Illness (CGI), the Patient Rated Global Improvement Scale of Sheehan, the Sheehan Clinician Rated Anxiety Scale, the Sheehan Patient Rated Anxiety Scale, Disability Scales, the Hamilton Anxiety Scale (HAM-A), the Phobia Scale (Sheehan after Marks and Mathews), and all questions contained therein and portions thereof. The above scales are published by the University of South Florida among others.

Panic Attacks

Certain embodiments of the methods of the present invention are of particular use in the measurement of panic attacks and their related symptoms, and in the measurement of the response of subjects experiencing panic attack symptoms to treatments, and in determining whether a drug composition is more effective in treating panic than a placebo composition.

Classical symptoms of a panic attack are as follows: chest pain or discomfort; choking; dizziness, unsteady feelings, or faintness; fear of dying; fear of going crazy or losing control; feelings of unreality, strangeness, or detachment from the environment; flushes or chills; nausea or abdominal distress; numbness or tingling sensations; palpitations or accelerated heart rate; shortness of breath or smothering sensation; sweating; and trembling or shaking. Classical panic attacks involve four or more of the above symptoms.

Described above are scales for measuring medical events or aspects thereof. Of particular utility in the present invention are scales wherein the score on the scale indicates the severity of a subject's panic symptoms, preferably those symptoms at the specific time (or only a few seconds, for example less than 300, 120, 100, 60, 30, 15, 10, or 5 seconds, before) the entry in the diary is made. Both scales that measure specific panic symptoms such as those above (or the severity thereof) and scales that measure panic symptoms overall or globally (or the severity thereof) are contemplated for use in the methods of the present invention.

Panic attacks are exceptionally common, affecting over 30% of the American population in a given year. Acute panic attacks may occur within any of number of well recognized psychiatric disorders, including panic disorder, generalized anxiety disorder, agoraphobia, specific phobias, and social phobia, or they may occur in patients who are otherwise well. Generally, panic attacks are self-limiting and people who suffer them recover fully and without developing panic disorder. Nevertheless, the panic attacks may be distressing and negatively impact quality of life, in particular by causing subjects experiencing the attacks to avoid the attack triggers. When the attack trigger involves a useful life function, such as driving over a bridge, the panic attacks can be quite detrimental. Despite their high prevalence, currently there are no medications approved in the U.S. for the treatment of acute panic attacks. This may reflect wholly or in part the absence of adequate means for measuring acute panic attacks as they occurred prior to the methods of the present invention disclosed herein.

Because adequate methods for measuring acute panic attacks as they occur, as opposed to post hoc, did not exist prior to the methods of the present invention, the natural duration of untreated panic attacks was unclear until now. Using methods of the present invention, we find panic attacks ranging from 8 minutes to more than 1 hour in patients with panic disorder, with the median panic attack duration for four patients studied in detail (see Example 1) being 30, 52, 22, and 11 minutes for each of the patients respectively.

Medical Events Appropriate for Recording using Methods of the Present Invention

The methods of the present invention can be used to record virtually any conceivable medical event, although they are of greatest use for medical events of a brief to moderate duration, for example medical events generally ranging in duration from 0.1 to 10,000 minutes, or from 0.5 to 1000 minutes, or from 1 to 100 minutes, or from 1 to 60 minutes. The durations of the medical events referred to above are not intended to imply that the medical event must be completely terminated within the indicated time interval, but merely imply that a sufficient portion of the medical event occurs in the indicated time interval that a useful record of the medical event can be made by recording within only that time interval.

The methods of the present invention are particularly useful for recording medical events, and especially medical events that occur outside of a monitored setting (e.g., preferably not medical events such as surgeries or other medical procedures that occur only in doctor's offices or hospitals). Panic attacks are medical events that have an optimal duration for recording using the methods of the present invention and that frequently occurs spontaneously, away from medical supervision. Exemplary other medical events for recording using the methods provided herein include cluster headaches, migraine headaches with or without aura, episodes of breakthrough pain, bouts of vertigo, allergy attacks, episodes of immobility associated with Parkinson's disease, cravings for nicotine or other drugs, cravings for food, episodes of agitation, congestive heart failure exacerbations, episodes of difficulty breathing, episodes of nausea, episodes of pelvic pain in interstitial cystitis, and overactive bladder.

Another type of medical event that is particularly useful to record using the methods of the present invention is the response of a subject to a medication, especially a medication self-administered by the patient outside of a monitored setting, e.g., a patient taking a pill or an inhalation off of an inhalation device at home or at work. When the effects of the medication come on rapidly, e.g. within 2, 3, 6, 12, 24, 48, or 60 minutes then the methods of the present invention are particularly advantageous for recording the onset of action of the medication and how the medication effects the subject in the initial time period after the subject takes the medication.

Measurement of the Effects of Medical Treatments

Certain methods of the present invention enable accurate determination of whether a medical treatment produces a therapeutic effect in a subject. Some such methods comprise (a) providing the subject an electronic diary, (b) administering the medical treatment to the subject, and (c) prompting the subject to enter in the diary at least one measure for symptoms being experienced by the subject at least twice within a 54 minute time period or at least twice within every 54 minute time period after administration of the medical treatment from the diary. Various time intervals between prompts and various durations over which at least a particular number of prompts (or less than a particular number of prompts) may be given are described in detail above. Further described above are means for determining when to begin prompting and when (if at all) to terminate prompting. The use of scales as a component of entries is also described above in detail. In certain embodiments, the subject records in the diary at least one measure of the symptoms prior to administration of the medical treatment. Such recording may be prompted by the diary or unprompted. Additionally, the method can further comprise the subject recording an entry in the diary, of at least one measure for symptoms being experienced by the subject, prior to administration of the medical treatment.

Other methods of the present invention enable measuring response of a subject experiencing panic attack symptoms to a treatment. Some such methods comprise (a) providing an electronic diary to a subject that experiences panic attack symptoms, (b) administering the treatment to the subject when the panic attack symptoms are present, and (c) entering in the diary at least one measure of the symptoms at least twice within 18 minutes or within every 18 minutes after administration of the treatment. Various time intervals between entries and various durations over which at least a particular number of entries may be made are described in detail above. The use of scales as a component of entries is also described above in detail. In certain methods, the score on a scale indicates a self-assessment by the subject of the severity of the subject's panic symptoms. Certain methods also involve the diary prompting the subject to make entries. Various time intervals between prompts and various durations over which at least a particular number of prompts (or less than a particular number of prompts) may be given are described in detail above. In certain embodiments entering of data occurs until deactivation of the diary or of the prompts. In certain embodiments, the subject records in the diary at least one measure of the symptoms prior to administration of the medical treatment. Such recording may be prompted by the diary or unprompted.

In terms of the nature of the medical treatment provided in the methods described above, the effects of virtually any medical treatment known in the art can be examined using the methods disclosed herein. In certain embodiments of the present invention, the medical treatment is administered by a doctor, nurse, other medical personnel, family member, co-worker, or the like. In certain preferred embodiments, the medical treatment is self-administered by the subject. In certain embodiments, the medical treatment is not a drug, for example, the therapy can be, for example, cognitive behavioral therapy, surgery, electrical brain stimulation, acupuncture, chiropractic care, massage, or the like. In certain embodiments, the medical treatment is a drug. In these embodiments, the drug can constitute virtually any chemical entity, be it small molecules (e.g., molecular weight less than 600 Daltons) or macromolecule (e.g., molecular weight greater than 1000 Daltons). Preferred, however, are drugs with sedative properties or that interact with or serve to modify the function of a neurotransmitter system in the brain of a mammal. Also preferred are drugs that modulate or otherwise interact with the GABA (gamma-aminobutyric acid) system in the brain of a subject, especially those drugs that serve to increase GABA-mediated neurotransmission or to mimic or otherwise augment the effects thereof. Particularly preferred are benzodiazepine drugs. Among benzodiazepine drugs, alprazolam and clonazepam are especially preferred.

Other specific treatments among those appropriate for testing using the methods of the present invention include the following:
(a) anti-psychotic drugs such as trifluoperazine or haloperidol, NSAID drugs such as naproxen or ketoprofen, triptans drugs such as sumatriptan, rizatriptan, prochlorperazine for the treatment of cluster headaches or migraine headaches;
(b) opioid drugs such as fentanyl, sufentanyl, remifentanyl, hydromorphone, oxymorphone, oxycodone, morphine, methadone, and meperidine for the treatment of episodes of breakthrough pain;
(c) drugs with anti-histamine properties such as meclizine or drugs with anti-cholinergic properties such as scopolamine for the treatment of bouts of vertigo;
(d) drugs with anti-histamine properties such as diphenhydramine, chlorpheniramine, or loratadine for the treatment of allergy attacks;
(e) drugs with pro-dopamine properties such as L-dopa, ropinirole, or pramipexole for the treatment of episodes of immobility associated with Parkinson's disease;
(f) drugs like nicotine or bupropion for the treatment of cravings for nicotine or other drugs;
(g) drugs like sibutramine or bupropion for the treatment of cravings for food;
(h) drugs with anti-psychotic or sedative properties like haloperidol, alprazolam, loxapine, or olanzapine for the treatment of episodes of agitation;
(i) drugs like loop diuretics such as furosemide or bumetanide for the treatment of congestive heart failure exacerbations;
(j) drugs like beta agonists such as albuterol or fomoterol for the treatment of episodes of difficulty breathing; and
(k) drugs with dopamine blocking properties like prochlorperazine, pro-gastric motility properties like metoclopramine, or serotonin blocking properties like ondansetron or granisetron for treatment of episodes of nausea.

In certain embodiments, the drug treatment is administered via any medically acceptable route of drug delivery. Exemplary routes of drug delivery include, but are not limited to, intranasally, intramuscularly, intravenously, orally, parenterally, transdermally, and rectally.

In certain embodiments, the drug is administered orally. Exemplary ways to accomplish oral administration of the drug include, but are not limited to, tablets, effervescent tablets, capsules, granulates, and powders. In certain embodiments, the drug is administered topically by mouth. Exemplary ways to accomplish topical administration include, but are not limited to, buccal tablets, sublingual tablets, drops, and lozenges. In certain embodiments, the drug is administered by injection. Exemplary types of injection include, but are not limited to, intravenous injection, intramuscular injection, and subcutaneous injection, for example by bolus injection or continuous intravenous infusion. In certain embodiments, the drug may be formulated in rectal compositions such as suppositories or retention enemas.

In certain preferred embodiments, the drug is administered by inhalation. In certain embodiments, administration by inhalation results in rapid drug absorption without the need for injection. In certain embodiments, the administration by inhalation of the drug is performed by administration of a composition to a patient in aerosol form such that the patient inhales the composition by mouth or endotracheal tube in the pulmonary tract. In certain embodiments, administration by inhalation is accomplished using an inhalation delivery device. In certain embodiments, administration by inhalation is accomplished using technology such as described in U.S. patent applications, Ser. Nos. 10/633,876 filed Aug. 4, 2003, and 10/633,877 filed Aug. 4, 2003. Exemplary inhalation delivery devices include, but are not limited to, nebulizers, metered-dose inhalers, dry-powder inhalers or other inhalers known to those skilled in the art.

Other exemplary inhalation devices are disclosed, e.g., in U.S. patent application Ser. Nos. 10/633,876 and U.S. Ser. No. 10/633,877, both filed on Aug. 4, 2003. Certain exemplary devices comprise a heat-conductive substrate onto which a film of drug is deposited. In certain embodiments, the surface area of the substrate is sufficient to yield a therapeutic dose of the drug aerosol when used by a subject. In certain embodiments, the desired dosage and selected drug film thickness dictate the minimum optimal substrate area in accord with the following relationship: film thickness (cm)×drug density (g/cm³)×substrate area (cm²)=dose (g).

In certain embodiments, the film of drug deposited on the substrate has a thickness of between about 0.05 μm and 20 μm. In certain embodiments, the film thickness for a given drug is such that drug-aerosol particles, formed by vaporizing the drug by heating the substrate and entraining the vapor in a gas stream, have (i) 10% by weight or less drug-degradation product, and (ii) at least 50% of the total amount of drug contained in the film. In certain instances, thinner drug films result in purer drug particles than thicker drug films. In certain embodiments, the structure and/or form of the drug are adjusted to increase aerosol purity and/or yield.

In certain embodiments, the drug compound is delivered as an aerosol. In certain embodiments, the mass median aerodynamic diameter (MMAD) of the aerosol particles is less than about 5 μm. In certain embodiments, the MMAD of the aerosol particles is less than about 3 μm. In certain embodiments, the MMAD is within a range of about 1 to 5 μm.

In certain embodiments, the medical treatment comprises a single drug compound. In certain embodiments, more than one drug compound is used. In certain treatments, the drug is used in a composition or separately administered with one or more additional compounds.

In certain embodiments, the dose of a drug to be delivered as a medical treatment of the present invention may be determined by dose escalation in subjects, until either an optimal therapeutic response is obtained or dose-limiting toxicity is encountered.

In certain embodiments, the actual effective amount of drug for a particular patient can vary according to at least one of the specific drug or combination of drugs being utilized; the particular composition formulated; the mode of administration; the age, weight, and condition of the patient; and the severity of the episode being treated.

In certain embodiments, the drug is delivered by a route of administration that results in peak plasma concentrations in the patient being obtained rapidly after initiation of administration of the drug to the patient. In certain embodiments, the peak plasma concentration is obtained within 54, 36, 24, 18, 15, 12, 10, 9, 6, 5, 3, 2, 1.5, or 1 minutes after initiation of drug administration.

In certain embodiments, the medical treatment provides rapid relief of symptoms being experienced by the user of the treatment. In certain embodiments, symptomatic improvement is achieved at a time point 54 minutes or less following initiation of administration of the treatment. In certain embodiments, improvement is achieved in the subject at a time point 18 minutes or less following initiation of administration of the drug. In certain embodiments, improvement is achieved at a time point 6 or 3 minutes or less following initiation of administration of the drug.

Comparisons of Treatments

Certain methods of the present invention enable determination of the relative effectiveness of different medical treatments. In certain embodiments, the methods assess the relative effectiveness of a first medical treatment and a second medical treatment and comprise the following:
(a) providing an electronic diary to a first subject that experiences one or more symptoms to be treated,
(b) administering the first medical treatment to the subject experiencing one or more symptoms to be treated,
(c) entering in the electronic diary at least one measure of symptoms at least twice within 54 minutes or at least twice within every 54 minute time period after administration of the medical treatment,
(d) repeating the above steps (a)-(c) for the second medical treatment with the first subject or a second subject,
(e) comparing diary entries made after administration of the first medical treatment with entries made after administration of the second medical treatment.

In certain embodiments, the methods enable determination of whether a drug composition is more effective in treating panic than a placebo composition and comprise the following:
(a) providing a diary to a first subject that has been identified as having had the presence of panic attack symptoms;
(b) administering to the first subject experiencing the panic attack symptoms the placebo composition;
(c) entering in the diary a score on a scale that measures panic attack symptoms at least three times every 18 minutes after administration of the placebo composition;
(d) repeating the above steps a-c for the drug composition with the first subject or with a second subject, and
(e) comparing the scores entered after administration of the drug composition with the scores entered after administration of the placebo composition and/or the duration of the event after administration of the drug composition with the duration of the event after administration of the of the placebo composition.

In some embodiments, the method comprises the further step of correlating a decrease in the duration of the event or in the scores of the symptoms after administration of the drug composition relative to the placebo composition with more effectiveness of the drug than the placebo in treating panic

Various time intervals between entries and various durations over which at least a particular number of entries are made are described in detail in sections above but apply also to the methods described in this section. Further described above but also applicable to the methods described in this section is the use of prompts to encourage appropriately frequent making of entries in the diary. Also described above are means for determining when to begin prompting and when (if at all) to terminate prompting. Also described above are means for determining that the subject is experiencing one or more symptoms and means for identifying the presence of panic attack symptoms in the subject. Such means commonly involve the subject experiencing the symptoms, noticing the symptoms, and alerting the diary to the presence of the symptoms, but automated means are also provided above. In terms of identifying the presence of panic attack symptoms in the subject, increased heart rate and increased respiratory minute volume (generally due primarily to increased tidal volume size without marked changes in respiratory rate) are physiological panic symptoms that may be detected in an automated fashion using instruments known in the art or described above.

The use of scales as a component of entries is also described above in detail, as are various types of medical treatments. Any or all of the medical treatment types described above may be compared using methods of the present invention. The methods described in this section may further comprise any scale or group of scales selected from scales known in the art or described above. In certain embodiments of the methods described in this section, the subject records in the diary at least one measure of the symptoms prior to administration of the medical treatments. Such recording may be prompted by the diary or unprompted.

The above methods may be used to compare treatments using any and all clinical trial designs known in the art, including crossover trial designs and non-crossover trial designs. In a crossover trial, a subject receives a first medical treatment on a particular occasion or over a particular period of time, and then the same subject receives a second medical treatment. This allows comparison of the effects of different medical treatments in the same subject. Preferably, multiple subjects are enrolled in such a trial, with some receiving the first medical treatment followed by the second medical treatment, and others receiving the second medical treatment followed by the first medical treatment. This approach reduces the chance that the order with which the medical treatments are delivered biases the results of the trail. In a non-crossover trial, each subject enrolled in the trial receives a single type of treatment, and multiple subjects must be enrolled in the trial in order to compare two or more different treatments.

The above methods may be used in blinded or non-blinded clinical trials. For blinded trials, the blinding may involve preventing the subjects receiving the treatments from knowing which treatment is which. The blinding may alternatively or in addition involve preventing subjects interacting with the subjects receiving the treatments from knowing which treatment is which. For example, the treatments may be delivered by doctors, nurses, pharmacists or the like in a coded fashion, wherein neither the subject receiving the treatment nor the medical personnel interacting with the patient know the code. Trials in which neither the treatment provider nor recipient knows which treatment is being given are known as double-blind trials. Such double-blind trials are preferred.

The above methods may be used in randomized or non-randomized clinical trials. For randomized trials, the subjects are randomly assigned to receive the first or the second medical treatment, or (in a cross-over design) are randomly assigned to receive the first or second medical treatment first, and the other treatment thereafter. Random assignment may also be done in trials in which more than two different treatment groups or treatments being compared.

The above methods may be used to compare two or more different medical treatments, or to compare one or more medical treatment to placebo. Thus, either the first medical treatment or the second medical treatment may be a placebo treatment.

The step of comparing the diary entries made after administration of the first medical treatment with entries made after administration of the second medical treatment may involve, especially for trials with relatively few participants (such as 5, 10, 20 or less), simply reading or otherwise reviewing the entries to qualitatively assess the relative effectiveness of the treatments. For trials involving greater numbers of participants, such as 20, 40, 80, 100, 200, 400 or more participants, it may be useful to use quantitative techniques in comparing the diary entries. In the cases where entries include scores, such as scores on a visual analogue scale, it may be useful to evaluate the mean, median, or mode scores at various points in time after treatment, or to evaluate changes in the scores after treatment (relative to, for example, scores on the same scale prior to treatment). With diary entries such as scores, the entries are themselves quantitative and thus can be analyzed using statistical methods directly. For example, one group of scores may be compared to another group of scores based on means using T-tests, or, without relying on means, using Mann-Whitney tests for non-paired group comparisons. Both parametric and non-parametric statistical tests are contemplated. Diary entries that are not quantitative per se can be changed to quantitative forms, for example “yes” can be coded as “1” and “no” as “0” or text diary entries can be read to make a (preferably blinded) quantitative assessment of the severity of symptoms at the time of the entry on, for example, a visual-analogue scale.

A key output of many statistical tests used to compare diary entries made after administration of the first medical treatment with entries made after administration of the second medical treatment is a “p-value.” P-values reflect the probability that differences observed between groups are due solely to chance. In certain embodiments of the methods of the present invention, the step of comparing involves obtaining a p-value indicative of the probability that the entries made after administration of the first medical treatment differ from those made after administration of the second medical treatment due solely to chance. The p-value obtained may be less than 0.1, 0.05, 0.01, 0.005, or 0.001 to indicate a statistically significant difference between the treatments.

In design of a clinical trial comparing two or more medical treatments involving use of statistical tests to compare the response to the treatments, it is often of value to plan in advance the number of subjects (subjects) required in the trial to achieve a statistically significant difference between the treatments given a particular difference in clinical response to the treatments. Such planning requires an up-front decision of the size of treatment effect (i.e., the magnitude of the difference between treatments) that the trial should be able to detect. The number of subjects then enrolled in the trial determines the power of the trial, i.e., the chance of detecting a statistically significant difference between the treatments. In certain methods of the present invention, the number of subjects enrolled in the trial (i.e. the number of subjects receiving the first and the second medical treatments) is selected such that a clinically relevant difference between the treatments has a greater than 50% probability of resulting in a p-value of less than 0.05 in the statistical test used to compare the treatments. In certain other embodiments, the trial is designed to have a greater than 25%, 60%, 70%, 80%, 90%, or 95% probability of yielding a p-value less than a particular desired p-value if there is a clinical relevant difference between the treatments. In certain other embodiments, the clinically relevant difference between the treatments for which the trial is designed is approximately the minimum clinically relevant difference. In other embodiments, the clinically relevant difference between the treatments for which the trial is designed exceeds the minimum clinically relevant difference.

Working Examples

The following working examples are meant to be illustrative, and are in no way intended to limit the scope of the invention.

### EXAMPLE 1

### Electronic Diary for Measurement of Panic Attacks

The electronic diary was a Patient Experience Diary (PED) programmed by invivodata, Inc. of Scotts Valley, Calif. (www.invivodata.com) implemented on a PalmPilot 515, manufactured by Palm Computing Inc. It had a touch-screen LCD display, and operated on a rechargeable lithium ion battery. The data captured on the PED was uploaded by modem to central server databases, where it was archived and analyzed.

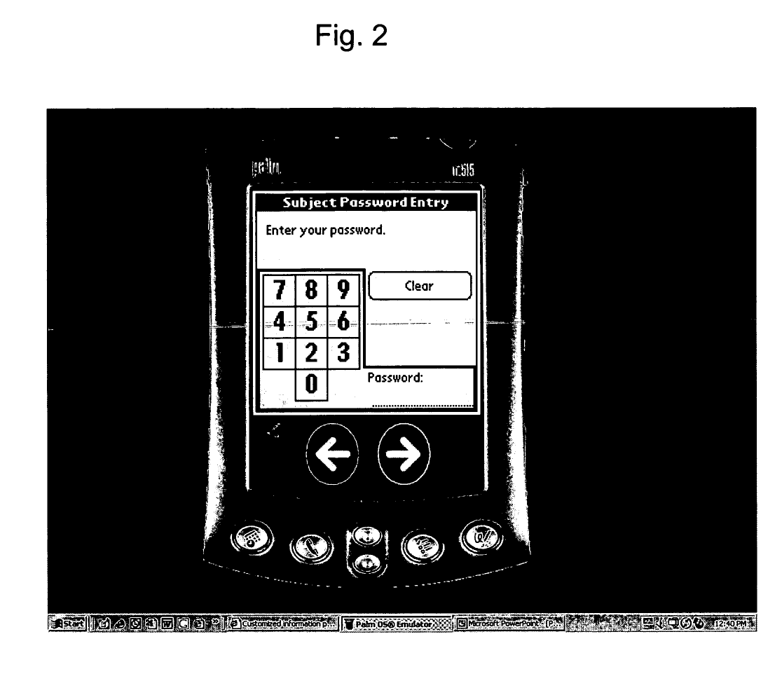

The PED had several ‘user-friendly’ features. Required actions were always specified on-screen in simple English, and all entries were made by tapping with a stylus directly on the screen. Options were presented in menus displayed with a simple and self-explanatory interface (see FIGS. 2 through 6).

The electronic diary was designed such that a subject (user or patient) would trigger the diary by pressing a button to indicate that the subject was experiencing panic symptoms. Then the diary would turn on (awake from a power-saving mode) and prompt the subject to complete a series of items as follows:

| Item # | Item Text | Response Options |
|---|---|---|
| 1 | Are you having a panic attack | Yes/No |
| | now? | |
| 2 | Enter your password | 4 digit numerical password |
| (shown in | | |
| FIG. 2) | | |
| 3 | How severe are your panic | 11-point visual analogue |
| (shown in | symptoms right now? | scale from “Not at all” to |
| FIG. 3) | | “Extremely” |
| 3 | RIGHT NOW | Ready to Take Med/Already |
| | | Taken Med/Don't Have Med |

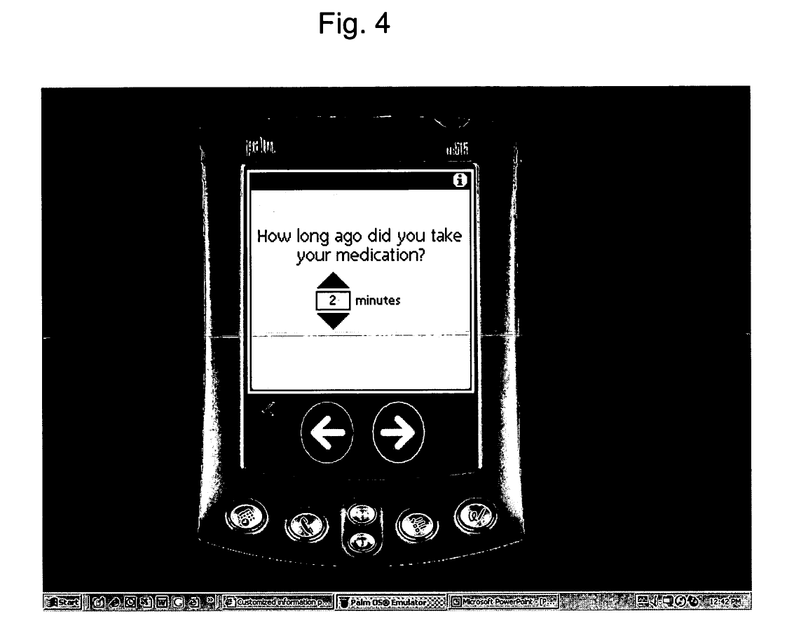

After the medication question, the diary did one of the following, depending of the response: if the response was “Ready to Take Med” the diary prompted the patient “Waiting . . . Please take your medication now. Tap -> when done.” Once the patient tapped on the forward arrow symbol at the bottom of the screen the timer began for the next assessment. If the response was “Already Taken Med” the diary then questioned “How long ago did you take your medication?” The diary then allowed the patient to answer any integer number between 1 and 60 minutes as shown in FIG. 4 and then the timer began for the next assessment. If the response was “Don't Have Med” the diary then reminded the patient “Remember to keep your medicine with you. Please tap ->” and once the patient tapped on the forward arrow symbol at the bottom of the screen the diary presented “Thank You, Goodbye” and turned off.

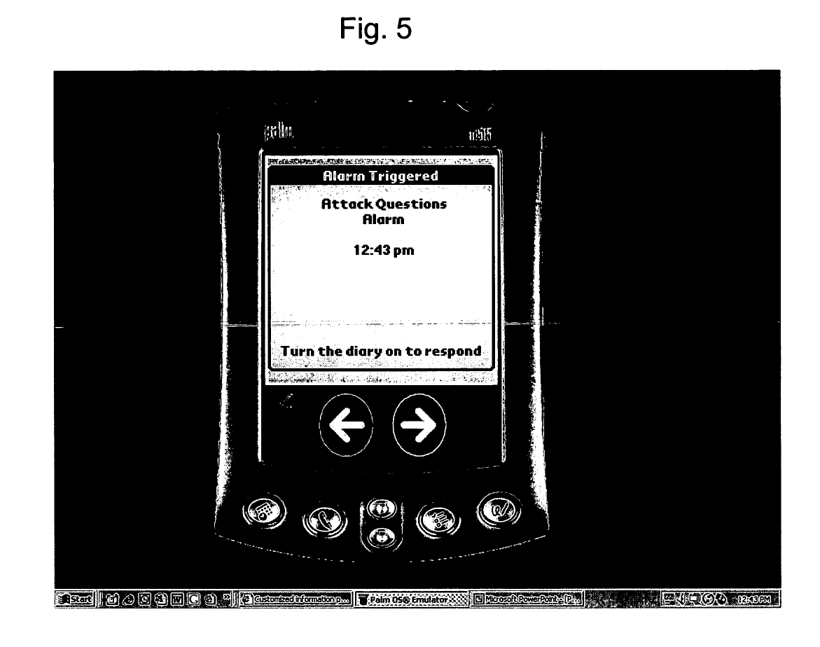

After completing the medication question, the diary went into a sleep mode until the diary's timer reached the pre-determined time (either 2 or 5 minutes, depending on how the diary was programmed) between diary prompts. Then the diary prompted the patient to make an entry by providing an auditory alarm that escalated through a series of tones beginning at a low volume, low frequency and ending at with fast tones at the Palm's maximum volume. The alarms started out beeping softly approximately once every two seconds, but then changed to more of a melody, so there was very little or no time between the tones for the remainder of the (up to three minute) alarm. In addition, the prompt consisted of text on the PED screen as shown in FIG. 5. The alarm ended when the patient turned on the diary. Once the patient turned the diary on, the patient was prompted as follows:

| Item # | Item Text | Response Options |
|---|---|---|
| 4 | How severe are your panic | 11-point visual analogue scale |
| (shown in | symptoms right now? | from “Not at all” to |
| FIG. 3) | | “Extremely” |

The diary then repeated the sleep mode-prompting alarm-item 4 cycle for a predetermined time interval until either 1 hour passed from the time of the first entry in the diary or until the patient responded “Not at all” three consecutive times when answering item 4. The diary then asked the patient the following additional item:

| Item # | Item Text | Response Options |
|---|---|---|
| 5 | Was you attack unexpected | Unexpected/Expected |
| | (out of the blue for no apparent | |
| | reason) or | |
| | situational/expected? | |

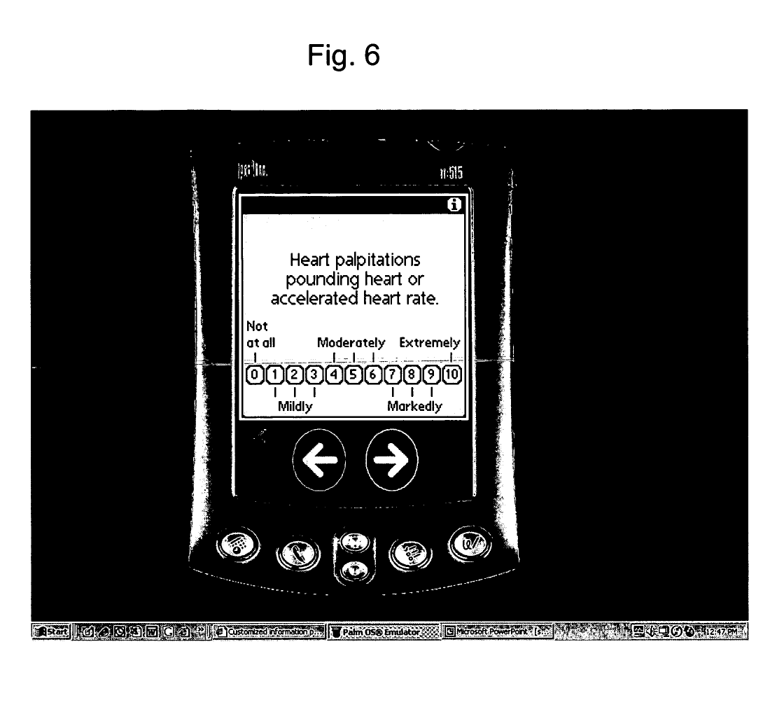

The diary then prompted the patient to do the following: “Please rate the following symptoms according to the greatest level of severity you experienced during the attack. Please tap ->.” After the patient tapped on the forward arrow symbol at the bottom of the screen, the diary then provided the following prompts:

| Item # | Item Text | Response Options |
|---|---|---|
| 6 | Heart palpitations, pounding | 11-point visual analogue scale from |
| (shown in FIG. 6) | heart, or accelerated heart rate. | “Not at all” to “Extremely” |
| 7 | Sweating | 11-point visual analogue scale from |
| | | “Not at all” to “Extremely” |
| 8 | Trembling or shaking | 11-point visual analogue scale from |
| | | “Not at all” to “Extremely” |
| 9 | Sensation of shortness of | 11-point visual analogue scale from |
| | breath or smothering | “Not at all” to “Extremely” |
| 10 | Feeling of choking | 11-point visual analogue scale from |
| | | “Not at all” to “Extremely” |
| 11 | Chest pain or discomfort | 11-point visual analogue scale from |
| | | “Not at all” to “Extremely” |
| 12 | Nausea or abdominal distress | 11-point visual analogue scale from |
| | | “Not at all” to “Extremely” |
| 13 | Dizzy, unsteady lightheaded or | 11-point visual analogue scale from |
| | faint | “Not at all” to “Extremely” |
| 14 | Feelings of unreality or being | 11-point visual analogue scale from |
| | detached from yourself | “Not at all” to “Extremely” |
| 15 | Fear of losing control or going | 11-point visual analogue scale from |
| | crazy | “Not at all” to “Extremely” |
| 16 | Fear or dying | 11-point visual analogue scale from |
| | | “Not at all” to “Extremely” |
| 17 | Numbness or tingling | 11-point visual analogue scale from |
| | sensations | “Not at all” to “Extremely” |
| 18 | Chills or hot flushes | 11-point visual analogue scale from |
| | | “Not at all” to “Extremely” |

The diary then concluded with the message “Thank you, Goodbye” and turned off.

### EXAMPLE 2

### Measurement of Panic Attacks in 5 Patients using an Electronic Diary

To determine the feasibility of using the methods described herein to record accurately medical events, even brief medical events involving panicking patients, the inventors conducted a pilot study of a patient experience diary for the assessment of the severity of panic attacks. Medical Director for the study was Roman J. Skowronski M.D., Ph. D. The study tested the electronic Patient Experience Diary described in Example 1 above. The study was conducted at 375 Municipal Drive, Suite 224, Richardson, Tex. 75080 by Principal Investigator Dr. Wayne C. Jones, M.D.

The objectives of the study were to determine the feasibility and applicability of using a patient experience diary (PED) for the real time assessment of severity of panic attacks in patients with panic disorder.

The overall study design was an open label, crossover design, single center, pilot study of Patient Experience Diary (PED) in patients with panic disorder to evaluate feasibility and applicability of the designed PED to assess in the real time severity of panic attacks. Five patients who experienced 2 and more panic attacks per week received the PED device programmed to record the severity of panic symptoms every 2 minutes for up to 60 minutes. The subjects used the PED to record the severity of panic attacks they experienced over a 2 week period. After two weeks they reported to the clinic where a survey concerning their evaluation of the PED was administered (survey described in greater detail below) and the PED was reset to a 5 minute reporting interval. Patients then used the PED again to record panic attacks occurring over the next 2 weeks.

The patients were five subjects with a history of panic attacks of 2 or more per week, of the following ages and genders: subject 1 (65 year old woman), subject 2 (67 year old woman), subject 3 (43 year old woman), subject 4 (70 year old man), subject 5 (48 year old man).

Prior to the initiation of the clinical study, the protocol, consent forms, and advertisements for subjects were reviewed and approved by the Institutional Review Board (IRB) of the participating study center. The study was conducted in accordance with the ethical principles that have their origin in the Declaration of Helsinki and were consistent with Good Clinical Practice and applicable regulatory requirements. The study was conducted in accordance with the regulations of the Food and Drug Administration as described in 21 CFR 50 and 56, applicable laws and the IRB requirements. Prior to study inception, each study participant read, signed and dated an IRB-approved consent form, explaining the nature, purpose, possible risks and benefits and the duration of the study. Patients were allowed to continue to take their usual medications at their usual doses throughout the study period. No medications were given as part of the study. Patients were instructed to indicate that they were ready to take their medication when they reached Item 3 in Example 1 above on the PED, even though they did not take any medication, as indicating an absence of medication would have resulted in the diary terminating its prompting of the patient and recording of the panic attack.

Prior to using the PED for field data collection, subjects were trained to use the PED's buttons as well as protocol-specific features. They were instructed to self-initiate a record of the beginning of each panic attack, after which time the PED prompted the subject to responds to the item “How severe are your panic symptoms right now?’ every 2 or every 5 minutes (depending on phase of the study) for 60 minutes. At the end of the 60 minute interval, or after the subject endorsed the ‘Not at all’ response to the above item, the PED prompted the subject to complete ratings of the greatest severity level experienced during the attack for 13 DSM IV criteria for panic attack. This series of questions was designed to be easy and quick to complete, as minimizing burden on the subject had been carefully considered. Subjects had the opportunity to practice the PED features and panic attack assessments until they were comfortable using the PED for field data collection.

Four out of five study participants (all except subject 5) successfully used the PED during the study to record more than one panic attack. The one participant who did not record any panic attacks commented that the diary was not sufficiently discreet for him to use, given that he was employed in a professional setting and his panic thus frequently occurred in settings where use of the diary would have been embarrassing.

The four participants who used the diary recorded a total of 38 panic attacks, 19 using the every 2 minute recording interval and 20 using the every 5 minute recording interval. Overall, 480 entries were made in the diaries, with 99.6% compliance of the subjects with the diary prompts (i.e., 99.6% of the time that the diary prompted a subject to make an entry, the subject did make an entry). This high compliance rate was remarkable, given the frequent prompting and the intense psychiatric symptoms being experienced by the patients.

The survey administered after each of the two, two-week periods of use of the diary asked the following questions with the following responses:
(a) Ease of use of the diary (0-5 scale, 5=very easy) mean response 4.8;
(b) Ease completing the diary assessments (0-5 scale; 5=very easy) mean response 4.9;
(c) Ease of responding to all of the diary alarms during an attack (0-5 scale; 5=very easy) mean response 4.0;
(d) Overall satisfaction with the diary (0-5 scale; 5=very satisified) mean response 4.8;
(e) Whether the patient would consider participating in another electronic diary clinical trial in the future (yes, no) response all four patients “yes” at both 2 and 5 minute prompting intervals;
(f) Familiarity of the patient prior to the study with computers (0-5 scale; 5 =very familiar) mean response 1.6;
(g) Familiarity of the patient prior to the study with handheld devices (ex. Palm device) (0-5 scale; 5=very familiar) mean response 1.0; and
(h) General comments (freeform)

The surveys indicated no particular preference of patients for the every 2 minute or every 5 minute recording intervals, indicating a remarkable ability of the patients to make frequent entries without difficulty

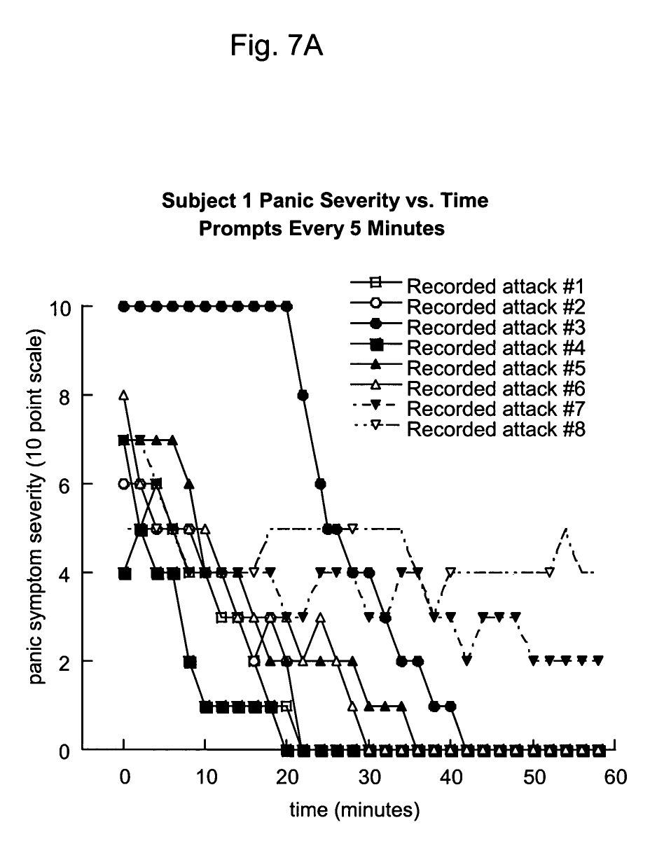

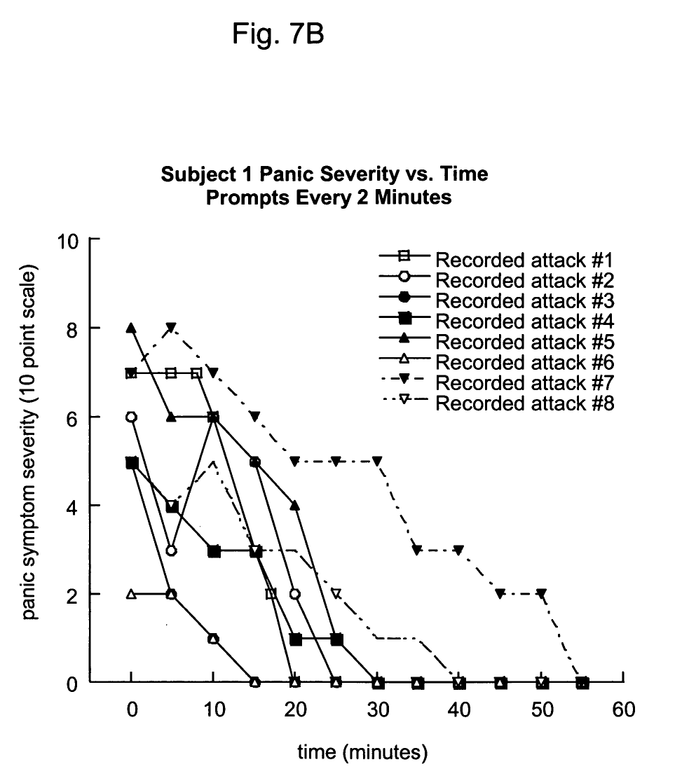

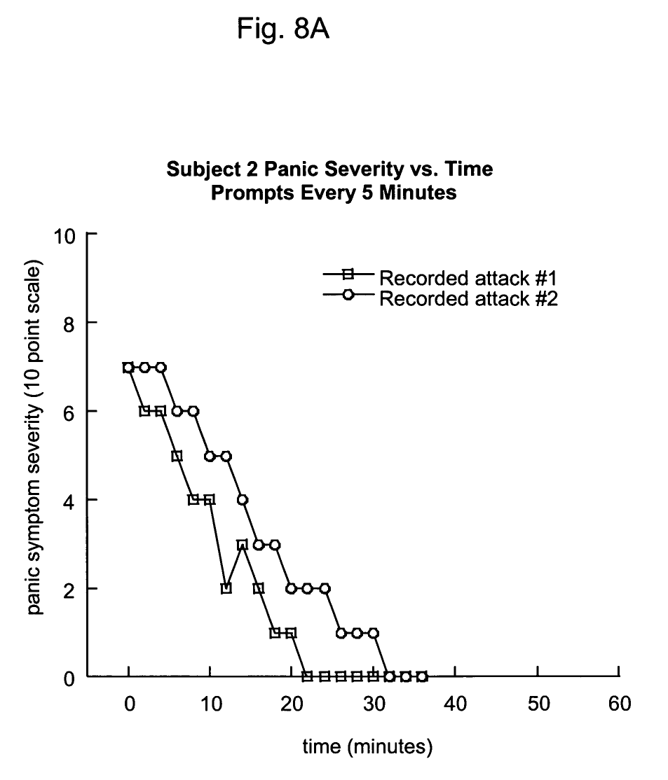

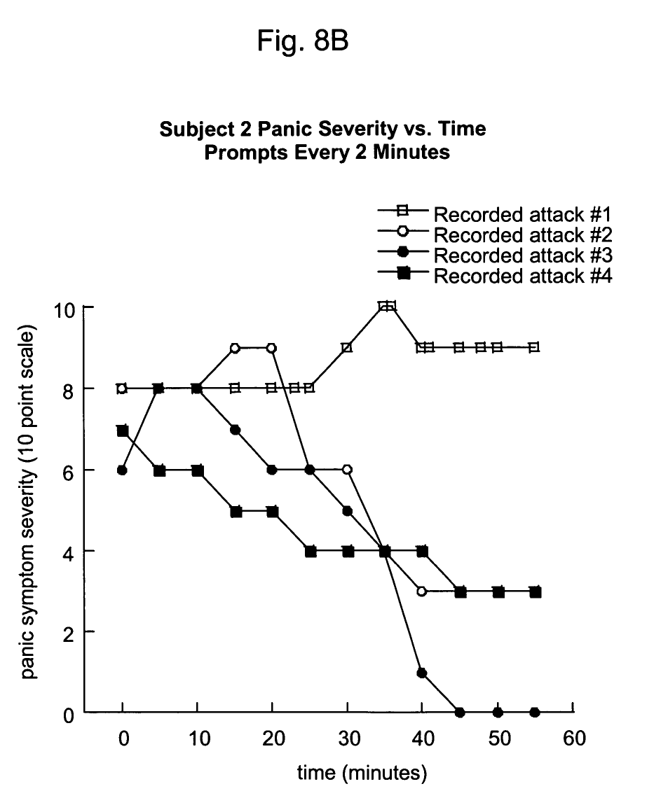

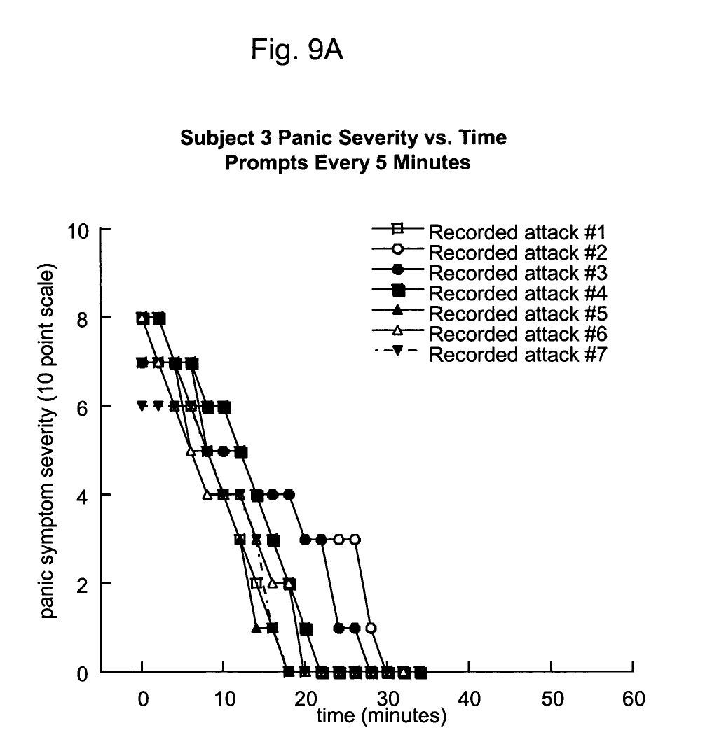

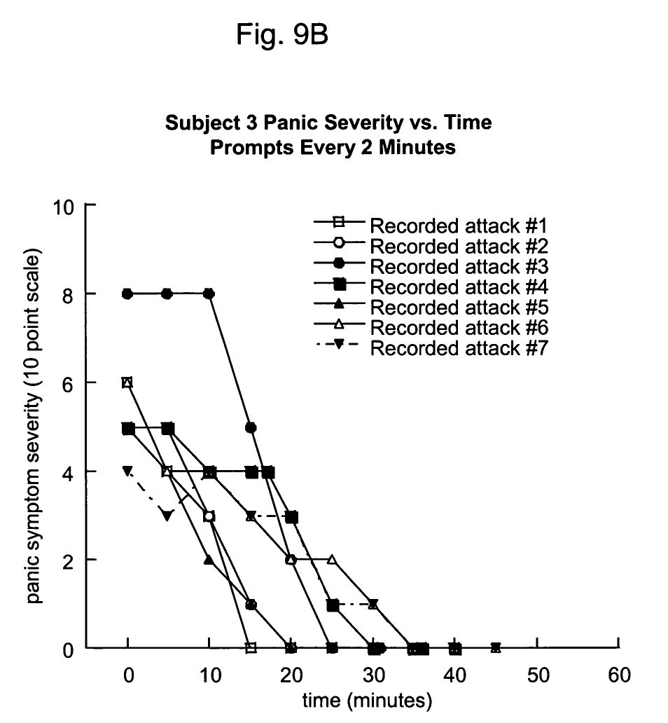

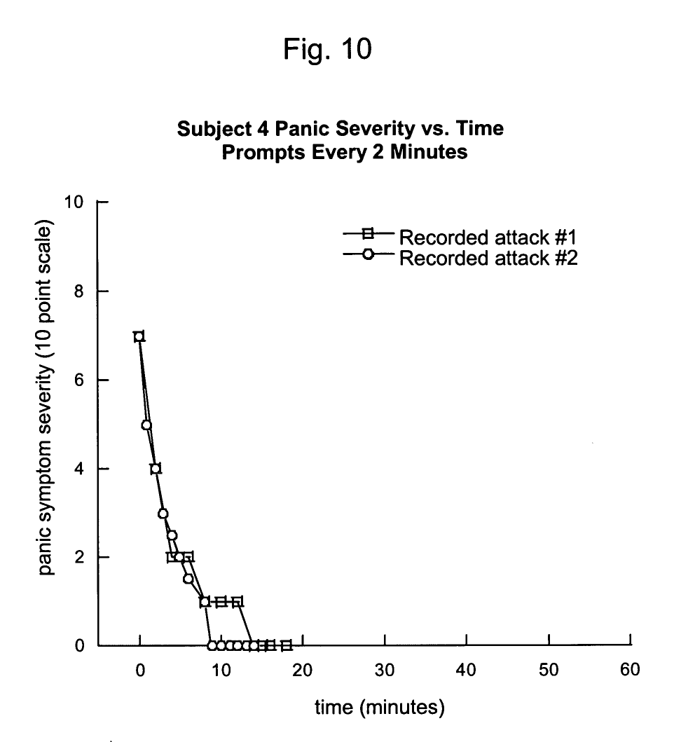

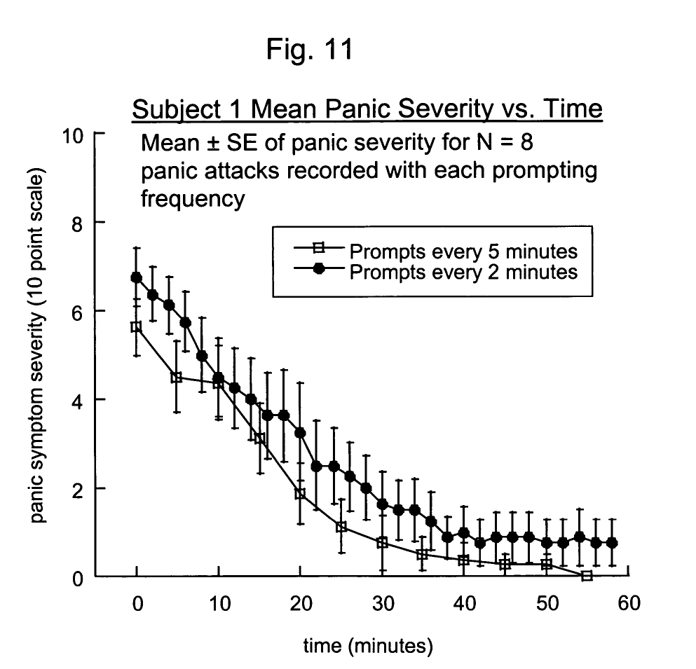

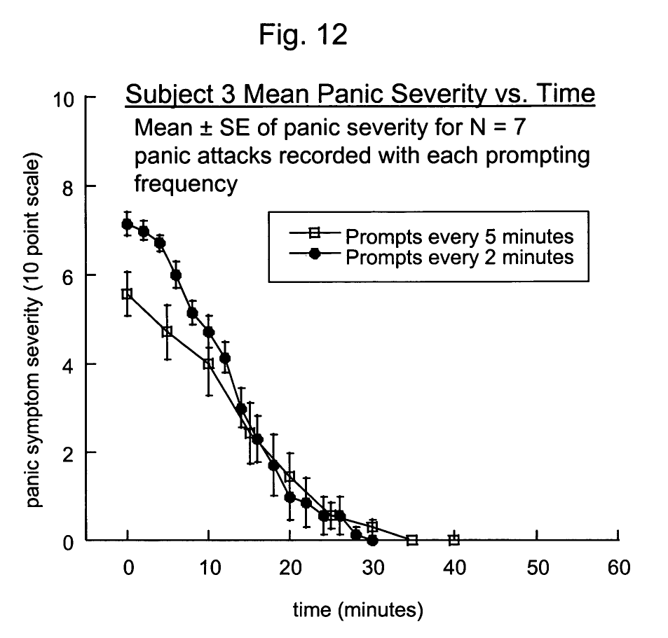

Graphs of the time course of panic severity during each recorded panic attack are provided in FIG. 7A for Subject 1 recording with prompts every 5 minutes, FIG. 7B for Subject 1 recording with prompts every 2 minutes, FIG. 8A for Subject 2 recording with prompts every 5 minutes, FIG. 8B for Subject 2 recording with prompts every 2 minutes, FIG. 9A for Subject 3 recording with prompts every 5 minutes, FIG. 9B for Subject 3 recording with prompts every 2 minutes, and FIG. 10 for Subject 4 recording with prompts every 2 minutes. Graphs of the average time course of panic (with error bars indicating the average score ± one standard error of the mean) for subjects 1 and 3 are provided in FIGS. 11 and 12 respectively, for both the 2 minute time interval between prompts and the 5 minute time interval between prompts. Of great importance is the similarity of responses under the 2 minute and 5 minute prompting conditions. A priori it seemed likely that the two different prompting conditions would result in very different responses, because the process of making such frequent entries would disrupt the natural history of the panic attack medical event, rendering the diary recording approach inaccurate. In contrast, the different prompting conditions resulted in virtually identical attack patterns, providing critical support for the validity of the recording method.

Prophetic Examples

The following prophetic example is meant to be illustrative, and is in no way intended to limit the scope of the invention.

### EXAMPLE 3

### Prophetic Determination of the Response of Patient Experiencing Panic Symptoms to Alprazolam

To determine the efficacy of alprazolam delivered by inhalation in the treatment of acute panic attacks, a clinical trial is conducted. The objectives of the trial are to determine the effectiveness of alprazolam delivered by inhalation at the time of onset of panic symptoms in decreasing the duration and/or severity of acute panic attacks in patients with panic disorder. The duration and severity of acute panic symptoms are measured using a patient experience diary (PED) for the real time assessment of severity of panic attacks. Patients are recruited for the study by posting in various public locations and medical clinics advertisements seeking patients who experience 2 or more panic attacks per week for participation in the study. To enroll patients who have not previously been treated for panic, the advertisement may include a description of panic symptoms, so that patients who experience panic attacks without being aware that such attacks constitute a medical condition are encouraged to volunteer.

The overall study design is a double-blind, randomized, placebo-controlled clinical trial. Patients 18-65 years of age reporting at least 2 panic attacks per week, without history of psychotic disorders, substance abuse disorders, dementia, or severe or life-threatening medical disorders such as serious heart or lung disease, eligible for the study. Patients not currently on medication for panic disorder, as well as patients currently using a stable dose of selective serotonin reuptake inhibitor (SSRI) the treatment of panic disorder are eligible for the study, as are patients currently taking alprazolam or other benzodiazepines on a PRN basis, if the patients agree not to use such drugs during the study period. Patients using chronic benzodiazepines or other sedative hypnotics for the treatment of panic disorder are excluded from the study, as are patients using psychotropic medications other than SSRIs or unstable SSRI doses. Eligible patients are enrolled in the study after being explained the risks and benefits of the study and signing informed consent.

The patients are then trained in use of the PED described in Examples 1 and 2 above, set to an every 3 minute prompting interval. After being trained in use of the PED, the patients then take the PED home for 3 weeks of use. During these 3 weeks, patients must record at least one panic attack per week in the diary of duration greater than 12 minutes (time from initial diary entry until the patient answers “not at all” to the item regarding the severity of their panic symptoms right now) to proceed to the randomization point in the study. Patients recording less than one panic attack per week, only very brief (less than 12 minute duration panic attacks), or having difficulty using the diary are dropped from the study. At this point, continuing patients are randomized to receive placebo inhaler, inhaler dispensing alprazolam 0.25 mg as an aerosol having an approximately 2 μm mass median aerodynamic diameter (MMAD), or inhaler dispensing alprazolam 0.5 mg as an aerosol having an approximately 2 μm MMAD. Patients are given four identical inhalers each, and instructed to use the inhalers at the time of their next 4 panic attacks (only 1 inhaler per attack). In particular, the patients are instructed to start the PED at the initiation of panic symptoms before taking their medication, to take the medication when prompted by the PED, and then to record the course of the panic attack after taking the medication as prompted by the PED.

Data from the 4 panic attacks during which the inhaler is used as panic treatment are recorded in the PED and transmitted to a central data storage site. The data is analyzed after each patient records 4 panic attacks, or after 8 weeks passes from the time the patient received the inhalers. For each patient, the following analyzed data is obtained: the mean duration of panic, the mean panic severity at 3, 6, 9, 12, 15, 18, 24, 30, 36, 42, 48, and 54 minutes following use of the inhaler, and the mean peak panic severity. Similar analyzed data are also obtained for each treatment group.

Typical results are as follows: the mean duration of panic is 30 minutes in patients receiving the placebo inhaler, 20 minutes in patients receiving alprazolam 0.25 mg, and 10 minutes in patients receiving alprazolam 0.5 mg. The mean panic severity at 12 minutes is 5 out of 10 in the patients receiving the placebo inhaler, 3 out of 10 in patients receiving alprazolam 0.25 mg, and 1.5 out of 10 in patients receiving alprazolam 0.5 mg. The mean peak panic severity is 7 in patients receiving the placebo inhaler, 6 in patients receiving alprazolam 0.25 mg, and 6 in patients receiving alprazolam 0.5 mg.

While the present invention has been described with reference to one or more particular variations, those skilled in the art will recognize that many changes may be made hereto without departing from the spirit and scope of the methods herein described and claimed.

1. A method of recording a medical event in real-time comprising providing a subject an electronic diary for recording one or more aspects of the medical event, wherein the diary electronically records a time at which entries to the diary are made without requiring the subject to input the time of each entry and wherein the diary further prompts the subject to make an entry in the diary at least twice within every 54 minute time period, following activation of the diary by the subject upon the initiation of the medical event, and continuing until deactivation of prompting from the diary.

2. The method of claim 1, wherein the diary prompts the subject to make an entry at least twice within every 18 minute time period.

3. The method of claim 2, wherein the diary prompts the subject to make an entry at least three times within every 18 minute time period.

4. The method of claim 1, wherein at least one aspect of the medical event is recorded on at least one scale.

5. The method of claim 4, wherein the diary prompts the subject to record at least one entry on the at least one scale at least two times within every 12 minute time period.

6. The method of claim 5, wherein the diary, at a pre-determined time interval of less than or equal to six minutes, prompts the subject to record at least one entry on the at least one scale.

7. The method of claim 6, wherein the diary, at a pre-determined time interval of less than or equal to every three minutes, prompts the subject to record at least one entry on at least one scale.

8. The method of claim 4, wherein the at least one scale comprises a visual-analogue scale.

9. The method of claim 8, wherein the visual-analogue scale has between 6 and 12 points.

10. The method of claim 9, wherein the visual-analogue scale measures the severity of panic.

11. A method of recording a panic attack in real-time comprising providing a subject an electronic diary for recording one or more aspect of the panic attack, wherein the subject records an entry in the diary at least three times within every 36 minute time period until deactivation of the diary.

12. The method of claim 11, wherein the subject experiencing the panic attack makes the entry in the diary.

13. The method of claim 12, wherein the at least three entries are made within every 18 minute time period.

14. The method of claim 12, wherein the diary prompts the subject to make entries in response to the subject activating the diary upon initiation of an attack.

15. The method of claim 14, wherein the diary prompts the subject to make an entry at a pre-determined time interval of less than or equal to every six minutes.

16. The method of claim 15, wherein the diary prompts the subject at a pre-determined time interval of less than or equal to every three minutes.

17. The method of claim 11, wherein the entry in the diary comprises a score on a scale.

18. The method of claim 17, wherein the entry in the diary comprises a score on a visual analogue scale.

19. The method of claim 18, wherein the visual-analogue scale has between 6 and 12 points.

20. The method of claim 19, wherein the visual-analogue scale has 11 points.

21. The method of claim 20, wherein the 11 points range from 0 to 10.

22. The method of claim 17, wherein the score indicates severity of the subject's panic symptoms when the entry is made in the diary.

23. The method of claim 22, wherein the diary automatically records a time when each entry is made in the diary.

24. A method of determining whether a medical treatment produces a therapeutic effect in a subject using real-time data comprising:
(a) providing the subject an electronic diary,
(b) administering the medical treatment to the subject, and
(c) prompting the subject to enter in the diary at least one measure for symptoms being experienced by the subject at least twice within every 54 minute time period after administration of the medical treatment until deactivation of prompting.

25. The method of claim 24, further comprising the subject recording an entry in the diary, of at least one measure for the symptoms being experienced, prior to administration of the medical treatment.

26. The method of claim 25, wherein the subject is prompted to make an entry at least once every 12 minutes after the administration of the medical treatment.

27. The method of claim 26, wherein the subject is prompted to make an entry at least three times every 24 minutes after administration of the medical treatment.

28. The method of claim 27, wherein the subject is prompted to make an entry at least once every six minutes.

29. The method of claim 28, wherein the subject is prompted to make an entry at least once every three minutes.

30. The method of claim 25, wherein the at least one measure of the symptoms comprises a score on a scale.

31. The method of claim 30, wherein the at least one measure of symptoms comprises a score on a visual analogue scale.

32. The method of claim 30, wherein the score indicates the severity of panic symptoms of the subject.

33. The method of claim 25, wherein the subject self administers the medical treatment.

34. The method of claim 24, wherein the medical treatment comprises administering a drug to a subject.

35. The method of claim 34, wherein the administering results in maximal plasma drug concentrations in the subject within 54 minutes.

36. The method of claim 34, wherein the drug is administered by inhalation.

37. The method of claim 36, wherein the drug is in the form of an aerosol characterized by a mass median aerodynamic diameter of less than 3 micrometers.

38. The method of claim 35, wherein the drug has sedative properties.

39. The method of claim 38, wherein the drug is a benzodiazepine.

40. The method of claim 39, wherein the drug is alprazolam.

41. A method of measuring response of a subject experiencing panic attack symptoms to a treatment in real-time comprising:
(a) providing the subject that experiences panic attack symptoms an electronic diary,
(b) administering the treatment to the subject when the panic attack symptoms are present, and
(c) entering in the diary at least one measure of the symptoms at least twice every 18 minutes after administration of the treatment until deactivation of the diary.

42. The method of claim 41, further comprising entering in the diary at least one measure of the panic attack symptoms prior to administration of the treatment.

43. The method of claim 41, further comprising entering in the diary at least one measure of the panic attack symptoms at least three times every 18 minutes after administration of the treatment.

44. The method of claim 43, wherein the diary prompts the subject to make an entry at least once every 6 minutes after administration of the treatment.

45. The method of claim 44, wherein the diary prompts the subject to make an entry at least once every 3 minutes after administration of the treatment.

46. The method of claim 41, wherein the at least one measure of the symptoms comprises a score on a scale.

47. The method of claim 46, wherein the at least one measure of symptoms comprises a score on a visual analogue scale.

48. The method of claim 47, wherein the visual analogue scale has between 6 and 12 points.

49. The method of claim 46, wherein the score indicates a self-assessment by the subject of the severity of the subject's panic symptoms.

50. The method of claim 41, wherein the subject self administers the treatment.

51. The method of claim 50, wherein the treatment comprises administering a drug to a subject.

52. The method of claim 51 wherein the drug is administered by inhalation.

53. The method of claim 52, wherein the drug is in the form of an aerosol characterized by a mass median aerodynamic diameter of less than 5 micrometers.

54. The method of claim 51, wherein the drug enhances GABA-type neurotransmission.

55. The method of claim 54, wherein the drug is a benzodiazepine.

56. The method of claim 55, wherein the drug is alprazolam.

57. The method of claim 41, wherein administration of the medical treatment occurs within 6 minutes of identification of the presence of panic attack symptoms.

58. A method of assessing the relative effectiveness of a first medical treatment and a second medical treatment comprising:
(a) providing an electron diary to a first subject that experiences one or more symptoms to be treated,
(b) administering the first medical treatment to the subject experiencing one or more of the symptoms to be treated,
(c) entering in the electronic diary in real-time at least one measure of symptoms at least twice every 54 minute time period after administration of the medical treatment until deactivation of the diary,
d) repeating the above steps (a)-(c) for the second medical treatment with the first subject or a second subject, and
e) comparing diary entries made after administration of the first medical treatment with entries made after administration of the second medical treatment.

59. The method of claim 58, further comprising recording in the diary at least one measure of the symptoms prior to administration of the medical treatments.

60. The method of claim 59, further comprising entering in the diary at least one measure of the symptoms at least three times every 18 minutes after administration of the medical treatments.

61. The method of claim 60, wherein the diary prompts the subject to make an entry at least once every 6 minutes after administration of the medical treatments.

62. The method of claim 61, wherein the at least one measure of the symptoms comprises a score on a scale.

63. The method of claim 62, wherein the at least one measure of symptoms comprises a score on a visual analogue scale.

64. The method of claim 62, wherein the score indicates the severity of panic symptoms.

65. The method of claim 62, wherein a subject who receives the first medical treatment also receives the second medical treatment.

66. The method of claim 62, wherein the first medical treatment comprises a placebo and the second medical treatment comprises a drug.

67. The method of claim 62, wherein subjects are randomly assigned to receive the first or the second medical treatment.

68. The method of claim 67, wherein comparing entries made in the diary after administration of the first medical treatment with entries made in the diary after administration of the second medical treatment comprises use of a statistical test.

69. The method of claim 68, wherein the number of subjects receiving the first and the second medical treatments is selected such that a clinically relevant difference between the treatments has a greater than 50% probability of resulting in a p-value of less than 0.05 in the statistical test.

70. A method of determining whether a drug composition is more effective in treating panic than a placebo composition comprising:
(a) providing a diary to a first subject that has been identified as having had the presence of panic attack symptoms;
(b) administering to the first subject experiencing the panic attack symptoms the placebo composition;
(c) entering in the diary a score on a scale that measures panic attack symptoms at least three times every 18 minutes after administration of the placebo composition until deactivation of the diary;
(d) repeating the above steps (a)-(c) for the drug composition with the first subject or with a second subject,
(e) comparing the scores entered after administration of the drug composition with the scores entered after administration of the placebo composition and/or the duration of the event after administration of the drug composition with the duration of the event after administration of the of the placebo composition, and
(f) correlating a decrease in the duration of the event or in the scores of the symptoms after administration of the drug composition relative to the placebo composition with more effectiveness of the drug than the placebo in treating panic.

71. The method of claim 70, further comprising repeating steps (a)-(f) for at least ten discrete first subjects receiving the placebo composition and at least ten discrete second subjects receiving the drug composition.

72. The method of claim 71, wherein comparing the scores entered after administration of the drug composition with the scores entered after administration of the placebo composition comprises determining whether the drug composition resulted in a statistically significantly different scores than the placebo composition.

73. The method of claim 72, wherein the drug composition comprises a benzodiazepine.

74. The method of claim 73, wherein the drug composition comprises alprazolam or clonazepam.

75. The method of claim 75, wherein administering the drug composition comprises the patient inspiring an aerosol.
